SEQUENCE LISTING (15 sequences)

SEQ ID NO: 1 - DNA, 171 bp, synthetic construct
  source 1..171: mol_type = other DNA; note = Nucleotide sequence of DNA library molecule
gaacgacatggctacgatccgacttnnnnnnnnnnnnnnnnnaagtcggaggccaagcggtcttaggaagacaannnnnnnnnnnnnnnnnnnnnnnnnctgataaggtcgccatgcctctcagtacgtcagcagttnnnnnnnnnnnnnnnnncaactccttggctcaca

SEQ ID NO: 2 - DNA, 24 bp, synthetic construct
  source 1..24: mol_type = other DNA; note = Nucleotide sequence of DNB primer
gccatgtcgttcggaaccgagtgt

SEQ ID NO: 3 - DNA, 36 bp, synthetic construct
  source 1..36: mol_type = other DNA; note = Nucleotide sequence of chip sequence synthesis primer
ctgctgacgtactgagaggcatggcgaccttatcag

SEQ ID NO: 4 - DNA, 36 bp, synthetic construct; identical to SEQ ID NO: 3
  source 1..36: mol_type = other DNA; note = Consensus sequence X1

SEQ ID NO: 5 - DNA, 13 bp, synthetic construct
  source 1..13: mol_type = other DNA; note = Consensus sequence X2
ttgtcttcctaag

SEQ ID NO: 6 - DNA, 44 bp, synthetic construct
  source 1..44: mol_type = other DNA; note = Nucleotide sequence of polyT primer
accgcttggcctccgttttttttttttttttttttttttttttv

SEQ ID NO: 7 - DNA, 36 bp, synthetic construct
  source 1..36: mol_type = other DNA; note = TSO sequence
aagcagtggtatcaacgcagagtacatnnnnnnggg

SEQ ID NO: 8 - DNA, 74 bp, synthetic construct
  source 1..74: mol_type = other DNA; note = Chip sequence
ctgctgacgtactgagaggcatggcgaccttatcagnnnnnnnnnnnnnnnnnnnnnnnnnttgtcttcctaag

SEQ ID NO: 9 - DNA, 21 bp, synthetic construct
  source 1..21: mol_type = other DNA; note = First bridging oligonucleotide sequence
ggggggggcttaggaagacaa

SEQ ID NO: 10 - DNA, 23 bp, synthetic construct
  source 1..23: mol_type = other DNA; note = Second bridging oligonucleotide sequence
ccccccccaagcagtggtatcaa

SEQ ID NO: 11 - DNA, 23 bp, synthetic construct
  source 1..23: mol_type = other DNA; note = Sequence of cDNA amplification primer 1
ctgctgacgtactgagaggcatg

SEQ ID NO: 12 - DNA, 22 bp, synthetic construct
  source 1..22: mol_type = other DNA; note = Sequence of cDNA amplification primer 2
accgcttggcctccgtgctatc

SEQ ID NO: 13 - DNA, 22 bp, synthetic construct
  source 1..22: mol_type = other DNA; note = Library construction primer 1
ctgctgacgtactgagaggcat

SEQ ID NO: 14 - DNA, 24 bp, synthetic construct
  source 1..24: mol_type = other DNA; note = Library construction primer 2
gagacgttctcgactcagaagatg

SEQ ID NO: 15 - DNA, 24 bp, synthetic construct
  source 1..24: mol_type = other DNA; note = Primer sequence to prepare DNB for sequencing
ggcctccgacttgagacgttctcg

(19) United States
(12) Patent Application Publication
(10) Pub. No.: US 2025/0163492 A1
(43) Pub. Date: May 22, 2025

(54) METHOD FOR GENERATING POPULATION OF LABELED NUCLEIC ACID MOLECULES AND KIT FOR THE METHOD

(71) Applicant: BGI SHENZHEN, Shenzhen (CN)

(72) Inventors: Xun XU, Shenzhen (CN); Ao CHEN, Shenzhen (CN); Wenwei ZHANG, Shenzhen (CN); Sha LIAO, Shenzhen (CN)

(21) Appl. No.: 18/722,541

(22) PCT Filed: Nov. 30, 2022
(86) PCT No.: PCT/CN2022/135363

§ 371 Date: Jun. 20, 2024

(30) Foreign Application Priority Data
Dec. 24, 2021 (CN) 202111602443.4

(51) Int. Cl. C12Q 1/6806; C12N 15/10; C12Q 1/6874

(52) U.S. Cl. CPC C12Q 1/6806; C12N 15/1065; C12Q 1/6874

(57) ABSTRACT

Provided are a method for performing position labeling of nucleic acid molecules, a method for constructing a nucleic acid molecule library for transcriptome sequencing, and a kit for implementing the method.

### TECHNICAL FIELD

The present application relates to the technical fields of transcriptome sequencing and biomolecular spatial information detection. Specifically, the present application relates to a method for positionally labeling nucleic acid molecules and a method for constructing a library of nucleic acid molecules for transcriptome sequencing. In addition, the present application also relates to a library of nucleic acid molecules constructed using the method, and a kit for implementing the method.

### Background Art

The spatial position of cells in tissues significantly affects their functions. To explore this spatial heterogeneity, it is necessary to quantify and analyze the genome or transcriptome of cells in combination with spatial coordinates of the cells. However, it is very laborious, expensive and has low precision to collect small tissue regions or even single cells for genome or transcriptome analysis. Therefore, it is necessary to develop a method that can achieve high-throughput detection of spatial information of biomolecules (e.g., localization, distribution, and/or expression of nucleic acids) at the single-cell level or even the sub-cellular level.

### Contents of the Application

The present application provides a new method for generating a population of labeled nucleic acid molecules, as well as a method for constructing a library of nucleic acid molecules based on this method and performing high-throughput sequencing.

### Method for Generating a Population of Labeled Nucleic Acid Molecules

In one aspect, the present application provides a method for generating a population of labeled nucleic acid molecules, which comprises the following steps:
(1) providing a biological sample and a nucleic acid array, wherein the nucleic acid array comprises a solid support, the solid support is coupled with multiple kinds of oligonucleotide probes, each kind of oligonucleotide probe comprises at least one copy, and the oligonucleotide probe comprises or consists of a consensus sequence X1, a tag sequence Y, and a consensus sequence X2 in the direction from 5′ to 3′, wherein, each kind of oligonucleotide probe has a different tag sequence Y, and the tag sequence Y has a nucleotide sequence unique to the position of the kind of oligonucleotide probe on the solid support;
(2) contacting the biological sample with the nucleic acid array so that the position of an RNA (e.g., mRNA) in the biological sample is mapped to the position of the oligonucleotide probe on the nucleic acid array; preprocessing the RNA (e.g., mRNA) in the biological sample to generate a first nucleic acid molecule population, wherein the preprocessing comprises:
(i) (a) using a primer A to perform reverse transcription of the RNA (e.g., mRNA) of the biological sample to generate a cDNA strand, the cDNA strand comprises a cDNA sequence complementary to the RNA (e.g., mRNA) and formed by reverse transcription primed by the primer A, and a 3′-end overhang; wherein, the primer A comprises a capture sequence A, the capture sequence A is capable of annealing to an RNA (e.g., mRNA) to be captured and initiating an extension reaction; and, (b) annealing a primer B to the cDNA strand generated in (a), and performing an extension reaction to generate a first extension product as a first nucleic acid molecule to be labeled, thereby generating a first nucleic acid molecule population; wherein, the primer B comprises a consensus sequence B, a complementary sequence of the 3′-end overhang, and optionally a tag sequence B; the complementary sequence of the 3′-end overhang is located at the 3′-end of the primer B; the consensus sequence B is located upstream of the complementary sequence of the 3′-end overhang (e.g., located at the 5′-end of the primer B); or,
(ii) (a) using a primer A′ to perform reverse transcription of the RNA (e.g., mRNA) of the biological sample to generate a cDNA strand; the cDNA strand comprises a cDNA sequence that is complementary to the RNA (e.g., mRNA) and formed by reverse transcription primed by the primer A′, and a 3′-end overhang; wherein, the primer A′ comprises a consensus sequence A and a capture sequence A, the capture sequence A is capable of annealing to an RNA (e.g., mRNA) to be captured and initiating an extension reaction; the consensus sequence A is located upstream of the capture sequence A (e.g., located at the 5′-end of the primer A′); (b) annealing a primer B′ to the cDNA strand generated in (a) and performing an extension reaction to generate a first extension product; wherein, the primer B′ comprises a consensus sequence B, a complementary sequence of the 3′-end overhang, and optionally a tag sequence B; the complementary sequence of the 3′-end overhang is located at the 3′-end of the primer B′; the consensus sequence B is located upstream of the complementary sequence of the 3′-end overhang (e.g., located at the 5′-end of the primer B′); and, (c) providing an extension primer to perform an extension reaction using the first extension product as a template to generate a second extension product as a first nucleic acid molecule to be labeled, thereby generating a first nucleic acid molecule population;
(3) generating a second nucleic acid molecule population from the first nucleic acid molecule population obtained in the previous step by a step selected from the following:
(i) annealing (e.g., in-situ annealing) the oligonucleotide probe to the first nucleic acid molecule to be labeled which is at the corresponding position of the oligonucleotide probe, by applying a annealing condition to the product of step (2), and performing an extension reaction to generate an extension product as a second nucleic acid molecule with a positioning tag, thereby generating a second nucleic acid molecule population; wherein the consensus sequence X2 or partial sequence thereof of the oligonucleotide probe is (a) capable of annealing to a complementary sequence or partial sequence thereof of the consensus sequence B of the first extension product obtained in step (2)(i), or, (b) capable of annealing to a complementary sequence or partial sequence thereof of the consensus sequence A of the second extension product obtained in step (2)(ii); or,
(ii) contacting a bridging oligonucleotide pair with the oligonucleotide probe and the first nucleic acid molecule population obtained in the previous step under a condition that allows annealing, annealing (e.g., in-situ annealing) the bridging oligonucleotide pair to the oligonucleotide probe and the first nucleic acid molecule to be labeled which is at the corresponding position of the oligonucleotide probe,
wherein, the bridging oligonucleotide pair is composed of a first bridging oligonucleotide and a second bridging oligonucleotide, and the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; and the second region of the first bridging oligonucleotide is capable of annealing to the consensus sequence X2 or partial sequence thereof of the oligonucleotide probe;
the second region of the second bridging oligonucleotide is (a) capable of annealing to a complementary sequence or partial sequence thereof of the consensus sequence B of the first extension product obtained in step (2)(i), or, (b) capable of annealing to a complementary sequence or partial sequence thereof of the consensus sequence A of the second extension product obtained in step (2)(ii);
wherein, among the bridging oligonucleotide pair to be contacted with the first nucleic acid molecule population and the oligonucleotide probe, the first bridging oligonucleotide and the second bridging oligonucleotide of the bridging oligonucleotide pair each exist in a single-stranded form, or, the first bridging oligonucleotide and the second bridging oligonucleotide of the bridging oligonucleotide pair are annealed to each other and exist in a partially double-stranded form;
performing a ligation reaction to ligate the nucleic acid molecule hybridized with the first region and the nucleic acid molecule hybridized with the second region of the same first bridging oligonucleotide, and/or, to ligate the nucleic acid molecule hybridized with the first region and the nucleic acid molecule hybridized with the second region of the same second bridging oligonucleotide; and performing an extension reaction to obtain a reaction product as a second nucleic acid molecule with a positioning tag, thereby generating a second nucleic acid molecule population; wherein, the ligation reaction and the extension reaction are performed in any order.

In certain embodiments, in step (3)(ii) of the method:
(1) the first region and the second region of the first bridging oligonucleotide are directly adjacent, the ligation of the nucleic acid molecule hybridized with the first region and the nucleic acid molecule hybridized with the second region of the same first bridging oligonucleotide comprises: using a nucleic acid ligase to ligate the nucleic acid molecule hybridized with the first region and the nucleic acid molecule hybridized with the second region of the same first bridging oligonucleotide; or,

The first bridging oligonucleotide comprises the first region, the second region and the third region between them, the ligation of the nucleic acid molecule hybridized with the first region and the nucleic acid molecule hybridized with the second region of the same first bridging oligonucleotide comprises: using a nucleic acid polymerase (e.g., a nucleic acid polymerase without 5′ to 3′ exonucleolytic activity or strand displacement activity) to perform a polymerization reaction with the third region as a template, and using a nucleic acid ligase to ligate the nucleic acid molecule hybridized with the first region and the nucleic acid molecule hybridized with the third region and the second region of the same first bridging oligonucleotide;
and/or
(2) the first region and the second region of the second bridging oligonucleotide are directly adjacent, the ligation of the nucleic acid molecule hybridized with the first region and the nucleic acid molecule hybridized with the second region of the same second bridging oligonucleotide comprises: using a nucleic acid ligase to ligate the nucleic acid molecule hybridized with the first region and the nucleic acid molecule hybridized with the second region of the same second bridging oligonucleotide; or,
the second bridging oligonucleotide comprises the first region, the second region and the third region between them, the ligation of the nucleic acid molecule hybridized with the first region and the nucleic acid molecule hybridized with the second region of the same second bridging oligonucleotide comprises: using a nucleic acid polymerase (e.g., a nucleic acid polymerase without 5′ to 3′ exonucleolytic activity or strand displacement activity) to perform a polymerization reaction with the third region as a template, and using a nucleic acid ligase to ligate the nucleic acid molecule hybridized with the first region and the nucleic acid molecule hybridized with the third region and the second region of the same second bridging oligonucleotide.

In certain embodiments, each kind of oligonucleotide probe comprises one copy.

In certain embodiments, each kind of oligonucleotide probe comprises multiple copies.

In certain embodiments, a region with one kind of oligonucleotide probe coupled to the solid support is referred to as a microdot. It is easy to understand that when each kind of oligonucleotide probe comprises one copy, each microdot is coupled with one oligonucleotide probe, and the oligonucleotide probes of different microdots have different tag sequences Y; when each kind of oligonucleotide probe comprises a plurality of copies, each microdot is coupled with a plurality of oligonucleotide probes of the same kind, the oligonucleotide probes in the same microdot have the same tag sequence Y, and the oligonucleotide probes in different microdots have different tag sequences Y.

In certain embodiments, the solid support comprises a plurality of microdots, each microdot is coupled with one kind of oligonucleotide probe, and each kind of oligonucleotide probe may comprise one or more copies.

In certain embodiments, the solid support comprises a plurality of (e.g., at least 10, at least 10², at least 10³, at least 10⁴, at least 10⁵, at least 10⁶, at least 10⁷, at least 10⁸, or more) microdots. In certain embodiments, the solid support comprises at least 10⁴ (e.g., at least 10⁴, at least 10⁵, at least 10⁶, at least 10⁷, at least 10⁸, at least 10⁹, at least 10¹⁰, at least 10¹¹, or at least 10¹²) microdots/mm².

In some embodiments, the spacing between adjacent microdots is less than 100 μm, less than 50 μm, less than 10 μm, less than 5 μm, less than 1 μm, less than 0.5 μm, less than 0.1 μm, less than 0.05 μm, or less than 0.01 μm.

In certain embodiments, the microdots have a size (e.g., equivalent diameter) of less than 100 μm, less than 50 μm, less than 10 μm, less than 5 μm, less than 1 μm, less than 0.5 μm, less than 0.1 μm, less than 0.05 μm, or less than 0.01 μm.

In certain embodiments, the method comprises step (1), step (2)(i) and step (3); wherein, in step (2)(i)(b), the primer B comprises the consensus sequence B, a complementary sequence of the 3′-end overhang, and the tag sequence B.

In some embodiments, the first extension product in step (2)(i)(b) comprises from 5′ to 3′: a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, in step (3), the second nucleic acid molecule derived from each copy of the same kind of oligonucleotide probe has a different tag sequence B as a UMI.

Embodiments Comprising Step (1), Step (2)(i) and Step (3)(i)

In certain embodiments, the method comprises step (1), step (2)(i) and step (3)(i); wherein the consensus sequence X2 or partial sequence thereof is capable of annealing to a complementary sequence or partial sequence thereof of the consensus sequence B; the extension product obtained in step (3)(i) is a labeled nucleic acid molecule, which comprises: a first strand comprising the sequence of the first nucleic acid molecule to be labeled, and/or, a second strand comprising the sequence of the oligonucleotide probe.

It is easy to understand that the “partial sequence of XX (sequence)” or “part of XX (sequence)” refers to a nucleotide sequence of at least one fragment of “XX (sequence)”.

For example, the consensus sequence X2 in its entire nucleotide sequence is capable of annealing to a complementary sequence or partial fragment thereof of the consensus sequence B. The consensus sequence X2 in its partial nucleotide sequence is also capable of annealing to a complementary sequence or partial fragment thereof of the consensus sequence B.

The “annealing” means that between two nucleotide sequences that anneal to each other, each base in one nucleotide sequence can be paired with a base in the other nucleotide sequence without mismatch or gap; or, between two nucleotide sequences that anneal to each other, most of bases in one nucleotide sequence can be paired with bases in the other nucleotide sequence, which allows for a mismatch or gap (e.g., a mismatch or gap of one or several nucleotides). That is, two nucleotide sequences capable of annealing to each other can be either completely complementary or partially complementary. The descriptions about “annealing” herein apply to the entire text of the present application unless otherwise indicated herein or otherwise clearly contradicted by context.

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, a complementary sequence of the tag sequence Y, and a complementary sequence of the consensus sequence X1.

In certain embodiments, the second strand comprises from 5′ to 3′: the consensus sequence X1, the tag sequence Y, the consensus sequence X2, the tag sequence B, a complementary sequence of the 3′-end overhang sequence, and a complementary sequence of the cDNA sequence that is complementary to the RNA and formed by reverse transcription primed by the primer A.

Embodiments Comprising Step (1), Step (2)(i) and Step (3)(i), for Generating the First Strand

In certain embodiments, the consensus sequence X2 or partial sequence thereof is capable of annealing to a complementary sequence or partial sequence thereof (e.g., 3′-end partial sequence thereof) of the consensus sequence B, and the complementary sequence of the consensus sequence B of the first extension product in step (2)(i) has a free 3′ end.

In certain embodiments, the extension product obtained in step (3)(i) is a labeled nucleic acid molecule, which comprises the first strand.

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, a complementary sequence of the tag sequence Y, and a complementary sequence of the consensus sequence X1.

In certain embodiments, in step (3)(i), the oligonucleotide probe is incapable of initiating an extension reaction (e.g., the 3′ end of the oligonucleotide probe is blocked).

In certain embodiments, in step (2)(i)(a) of the method, the capture sequence A of the primer A is a random oligonucleotide sequence.

In some embodiments, the first extension product in step (2)(i)(b) comprises from 5′ to 3′: a cDNA sequence that is complementary to the RNA and formed by reverse transcription primed by the primer A, the 3′-end overhang sequence, a complementary sequence of the tag sequence B, and a complementary sequence of the consensus sequence B.

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, a complementary sequence of the tag sequence Y, and a complementary sequence of the consensus sequence X1.

In certain embodiments, in step (2)(i)(a) of the method, the capture sequence A of the primer A is a poly(T) sequence or a specific sequence targeting a target nucleic acid.

In certain embodiments, the primer A also comprises a consensus sequence A, and optionally a tag sequence A, such as a random oligonucleotide sequence.

In certain embodiments, the capture sequence A is located at the 3′-end of the primer A.

In certain embodiments, the consensus sequence A is located upstream of the capture sequence A (e.g., located at the 5′-end of the primer A).

In certain embodiments, the first extension product in step (2)(i)(b) comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the first strand comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, a complementary sequence of the consensus sequence B, a complementary sequence of the tag sequence Y, and a complementary sequence of the consensus sequence X1.

Embodiments Comprising Step (1), Step (2)(i) and Step (3)(i), for Generating the Second Strand

In certain embodiments, the consensus sequence X2 or partial sequence thereof (e.g., 3′-end partial sequence thereof) is capable of annealing to a complementary sequence or partial sequence thereof of the consensus sequence B, and the consensus sequence X2 of the oligonucleotide probe has a free 3′ end.

In certain embodiments, the extension product obtained in step (3)(i) is a labeled nucleic acid molecule, which comprises the second strand.

In certain embodiments, the second strand comprises from 5′ to 3′: the consensus sequence X1, the tag sequence Y, the consensus sequence X2, the tag sequence B, a complementary sequence of the 3′-end overhang sequence, and a complementary sequence of the cDNA sequence that is complementary to the RNA and formed by reverse transcription primed by the primer A.

In certain embodiments, the first extension product obtained in step (2)(i) is incapable of initiating an extension reaction (e.g., the 3′ end of the first extension product obtained in step (2)(i) is blocked).

In certain embodiments, in step (2)(i)(a) of the method, the capture sequence A of the primer A is a random oligonucleotide sequence.

In some embodiments, the first extension product in step (2)(i)(b) comprises from 5′ to 3′: a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the second strand comprises from 5′ to 3′: the consensus sequence X1, the tag sequence Y, the consensus sequence X2, the tag sequence B, a complementary sequence of the 3′-end overhang sequence, a complementary sequence of the cDNA sequence that is complementary to the RNA and formed by reverse transcription primed by the primer A.

In certain embodiments, in step (2)(i)(a) of the method, the capture sequence A of the primer A is a poly(T) sequence or a specific sequence targeting a target nucleic acid.

In certain embodiments, the primer A also comprises a consensus sequence A, and optionally a tag sequence A, such as a random oligonucleotide sequence.

In certain embodiments, the capture sequence A is located at the 3′-end of the primer A.

In certain embodiments, the consensus sequence A is located upstream of the capture sequence A (e.g., located at the 5′-end of the primer A).

In certain embodiments, the first extension product in step (2)(i)(b) comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the second strand comprises from 5′ to 3′: the consensus sequence X1, the tag sequence Y, the consensus sequence X2, the tag sequence B, a complementary sequence of the 3′-end overhang sequence, a complementary sequence of the cDNA sequence that is complementary to the RNA and formed by reverse transcription primed by the primer A, optionally a complementary sequence of the tag sequence A, a complementary sequence of the consensus sequence A.

As used herein, the term “UMI” refers to “Unique Molecular Identifier,” which can be used to characterize and/or quantify a nucleic acid molecule. Unless otherwise indicated herein or clearly contradicted by context, the present application does not limit the location and quantity of the UMI or complementary sequence thereof in the nucleic acid molecule. For example, when the cDNA strand comprises the UMI or complementary sequence thereof, the UMI or complementary sequence thereof can be located 3′ of the cDNA sequence in the cDNA strand, or can be located 5′ of the cDNA sequence, or the UMI or complementary sequence thereof can be comprised both 3′ and 5′ of the cDNA sequence. When a complementary strand of the cDNA strand comprises the UMI or complementary sequence thereof, the UMI or complementary sequence thereof can be located 3′ of the complementary sequence of the cDNA sequence in the complementary strand of the cDNA strand, or can also be located 5′ of the complementary sequence of the cDNA sequence, or the UMI or complementary sequence thereof can be comprised both 3′ and 5′ of the cDNA sequence. In certain embodiments, the UMI can be introduced through the primer A, and/or through the primer B. In certain embodiments, the UMI can be introduced through the primer A′, and/or through the primer B′.

An exemplary embodiment of the present application comprising step (1), step (2)(i) and step (3)(i) is described in detail as follows:

I. An exemplary embodiment for preparing a cDNA strand comprising a complementary sequence of UMI close to the 3′-end by using an RNA (e.g., mRNA) in the sample as a template comprises the following steps (as shown in FIG. 2):

(1) using a reverse transcriptase (e.g., a reverse transcriptase with terminal deoxynucleotidyl transferase activity) and a primer A to perform reverse transcription of the RNA molecule (e.g., mRNA molecule) in the permeabilized sample to generate a cDNA, and adding an overhang (e.g., an overhang comprising three cytosine nucleotides) to the 3′-end of the cDNA. The reverse transcription reaction can be carried out by using various reverse transcriptases with terminal deoxynucleotidyl transferase activity. In certain preferred embodiments, the reverse transcriptase used does not have RNase H activity.

In certain embodiments, the primer A comprises a poly(T) sequence and a consensus sequence A (CA). Normally, the poly(T) sequence is located at the 3′-end of the primer A to initiate reverse transcription.

In certain embodiments, the primer A comprises a random oligonucleotide sequence, and can be used to capture an RNA without a poly(A) tail. Typically, the random oligonucleotide sequence is located at the 3′-end of the primer A to initiate reverse transcription.

(2) using a primer B to anneal to or hybridize with the cDNA strand. The primer B comprises a consensus sequence B (CB), a unique molecular identifier (UMI) sequence, and a complementary sequence of the 3′-end overhang of the cDNA. Subsequently, the nucleic acid fragment that hybridizes with or anneals to the primer B can be extended under the presence of a nucleic acid polymerase by using the UMI sequence and the consensus sequence B as templates, thereby generating a nucleic acid molecule, which comprises a complementary sequence of the UMI sequence and a complementary sequence of the consensus sequence B at the 3′-end.

Typically, the consensus sequence B is located upstream of the UMI sequence (e.g., located 5′ of the UMI sequence), and the sequence complementary to the 3′-end overhang of the cDNA strand is located at the 3′-end of the primer B.

For example, when the cDNA strand comprises an overhang of three cytosine nucleotides at the 3′-end, the primer B may comprise GGG at its 3′-end. In addition, the nucleotides of the primer B can also be modified (e.g., the primer B can be modified to comprise a locked nucleic acid) to enhance the binding affinity for the complementary pairing between the primer B and the 3′-end overhang of the cDNA strand.

Without being limited by any theory, various suitable nucleic acid polymerases (e.g., DNA polymerase or reverse transcriptase) can be used to perform the extension reaction, as long as they can use the sequence of the primer B or partial sequence thereof as a template to extend the annealed or hybridized nucleic acid fragment (reverse transcription product). In certain exemplary embodiments, the annealed or hybridized nucleic acid fragment (reverse transcription product) can be extended by using the same reverse transcriptase used in the aforementioned reverse transcription step.

In some embodiments, this step is performed simultaneously with step (1) (e.g., in the same reaction system).

In certain embodiments, the method optionally further comprises step (3): adding RNase H to digest the RNA strand in the RNA/cDNA hybrid to form a cDNA single strand.

In certain embodiments, the method does not comprise the step (3).

An exemplary structure of the cDNA strand prepared by the above exemplary embodiment comprises: the consensus sequence A, the cDNA sequence, the 3′-end overhang sequence, a complementary sequence of the UMI sequence, and a complementary sequence of the consensus sequence B.

II. An exemplary embodiment of labeling the 3′-end of a cDNA strand with a complementary sequence of an oligonucleotide probe (also known as chip sequence) to form a new nucleic acid molecule comprising the chip sequence information (i.e., a nucleic acid molecule labeled with the chip sequence) comprises the following steps (shown in FIG. 4):

In some embodiments, the consensus sequence X2 or partial sequence thereof of the chip sequence is capable of annealing to a complementary sequence or partial sequence thereof of the consensus sequence B of the cDNA strand obtained in the above step I. Thereby a new nucleic acid molecule comprising the chip sequence information (i.e., a nucleic acid molecule labeled with the chip sequence) can be obtained by annealing or hybridizing the cDNA strand to the chip sequence, and performing an extension reaction under the presence of a polymerase.

An exemplary structure of the new nucleic acid molecule comprising the chip sequence information formed by the above exemplary embodiment comprises a nucleic acid strand and/or a complementary nucleic acid strand thereof, wherein the nucleic acid strand comprises from 5′ to 3′: the consensus sequence A, the cDNA sequence, the 3′-end overhang sequence, a complementary sequence of the UMI sequence, a complementary sequence of the consensus sequence B, a complementary sequence of the tag sequence Y, and a complementary sequence of the consensus sequence X1.

Embodiments Comprising Step (1), Step (2)(i) and Step (3)(ii)

In certain embodiments, the method comprises step (1), step (2)(i) and step (3)(ii); wherein, the second region of the second bridging oligonucleotide is capable of annealing to a complementary sequence or partial sequence thereof of the consensus sequence B of the first extension product obtained in step (2)(i); the reaction product obtained in step (3)(ii) is a labeled nucleic acid molecule, which comprises: a first strand comprising the sequence of the first nucleic acid molecule to be labeled, and/or a second strand comprising the sequence of the oligonucleotide probe.

It is easy to understand that the second region of the second bridging oligonucleotide is capable of annealing to a complementary sequence or partial sequence thereof of the consensus sequence B of the first extension product obtained in step (2)(i).

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, the tag sequence B, a complementary sequence of the 3′-end overhang sequence, a complementary sequence of the cDNA sequence that is complementary to the RNA and formed by reverse transcription primed by the primer A.

Embodiments Comprising Step (1), Step (2)(i) and Step (3)(ii), for Generating the First Strand

In certain embodiments, the second region of the second bridging oligonucleotide is capable of annealing to a complementary sequence or partial sequence thereof (e.g., 3′-end partial sequence thereof) of the consensus sequence B of the first extension product obtained in step (2)(i), and the second region of the first bridging oligonucleotide has a free 3′ end.

In certain embodiments, the reaction product obtained in step (3)(ii) is a labeled nucleic acid molecule, which comprises the first strand.

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, the second region of the first bridging oligonucleotide is located at the 3′-end of the first bridging oligonucleotide.

In certain embodiments, the first region of the first bridging oligonucleotide is located at the 5′-end of the first bridging oligonucleotide.

In certain embodiments, the first bridging oligonucleotide does not comprise the third region, and/or the second bridging oligonucleotide does not comprise the third region.

In certain embodiments, the first bridging oligonucleotide comprises a 5′ phosphate at the 5′-end.

In certain embodiments, the first bridging oligonucleotide comprises a free —OH at the 3′-end.

In certain embodiments, in step (3)(ii), the second bridging oligonucleotide is incapable of initiating an extension reaction (e.g., the 3′end of the second bridging oligonucleotide is blocked), and/or, the oligonucleotide probe is incapable of initiating an extension reaction (e.g., the 3′-end of the oligonucleotide probe is blocked).

In certain embodiments, in step (2)(i)(a) of the method, the capture sequence A of the primer A is a random oligonucleotide sequence.

In certain embodiments, the first extension product in step (2)(i)(b) of the method comprises from 5′ to 3′: a cDNA sequence that is complementary to the RNA and formed by reverse transcription primed by the primer A, the 3′-end overhang sequence, a complementary sequence of the tag sequence B, and a complementary sequence of the consensus sequence B.

In some embodiments, the first strand comprises from 5′ to 3′: a cDNA sequence that is complementary to the RNA and formed by reverse transcription primed by the primer A, the 3′-end overhang sequence, a complementary sequence of the tag sequence B, a complementary sequence of the consensus sequence B, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, in step (2)(i)(a), the capture sequence A of the primer A is a poly(T) sequence or a specific sequence targeting a target nucleic acid.

In certain embodiments, the primer A also comprises a consensus sequence A, and optionally a tag sequence A, such as a random oligonucleotide sequence.

In certain embodiments, the capture sequence A is located at the 3′-end of the primer A.

In certain embodiments, the first extension product in step (2)(i)(b) comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the first strand comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, a complementary sequence of the consensus sequence B, optionally a complementary sequence of the third region of the second bridging oligonucleotide, the first bridging oligonucleotide sequence, a complementary sequence of the tag sequence Y, and a complementary sequence of the consensus sequence X1.

It is easy to understand that in step (3)(ii), after the first bridging oligonucleotide and the second bridging oligonucleotide are annealed to the oligonucleotide probe and the first nucleic acid molecule to be labeled which is at the corresponding position of the oligonucleotide probe, the ligation reaction for ligating the nucleic acid molecule hybridized with the first region and the nucleic acid molecule hybridized with the second region of the same first bridging oligonucleotide, and/or, ligating the nucleic acid molecule hybridized with the first region and the nucleic acid molecule hybridized with the second region of the same second bridging oligonucleotide, and the extension reaction in step (3)(ii) can be performed in any order, as long as a second nucleic acid molecule with a positioning tag can be obtained.

For example, when the ligation reaction and the extension reaction are carried out in the same system, the first strand can be obtained by ligating the nucleic acid molecule hybridized with the first region and the nucleic acid molecule hybridized with the second region of the same second bridging oligonucleotide, and extending the first bridging oligonucleotide by an extension reaction. In this case, the polymerase used in the extension reaction preferably does not have strand displacement activity or 5′ to 3′ exonucleolytic activity.

For example, when the ligation reaction and the extension reaction are performed in different systems, the ligation reaction is performed followed by the extension reaction, the first strand can be obtained in the following exemplary manner:
(A) ligating the nucleic acid molecule hybridized with the first region and the nucleic acid molecule hybridized with the second region of the same second bridging oligonucleotide, and extending the first bridging oligonucleotide by an extension reaction to obtain the first strand; wherein, the polymerase used for the extension reaction preferably has or does not have strand displacement activity or 5′ to 3′ exonucleolytic activity;
or,
(B) ligating the nucleic acid molecule hybridized with the first region and the nucleic acid molecule hybridized with the second region of the same first bridging oligonucleotide, and extending the first nucleic acid molecule to be labeled by an extension reaction to obtain the first strand; wherein, the polymerase used for the extension reaction preferably has strand displacement activity or 5′ to 3′ exonucleolytic activity.

For example, when the ligation reaction and the extension reaction are performed in different systems, and the extension reaction is performed followed by the ligation reaction, the first strand can be obtained by extending the first bridging oligonucleotide by an extension reaction, and then ligating the nucleic acid molecule hybridized with the first region and the nucleic acid molecule hybridized with the second region of the same second bridging oligonucleotide. In this case, the polymerase used in the extension reaction preferably does not have strand displacement activity or 5′ to 3′ exonucleolytic activity.

Embodiment Comprising Step (1), Step (2)(i) and Step (3)(ii), for Generating the Second Strand

In certain embodiments, the second region of the second bridging oligonucleotide is capable of annealing to a complementary sequence or partial sequence thereof of the consensus sequence B of the first extension product obtained in step (2)(i), and the second region of the second bridging oligonucleotide has a free 3′ end.

In certain embodiments, the reaction product obtained in step (3)(ii) is a labeled nucleic acid molecule, which comprises the second strand.

In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, the tag sequence B, a complementary sequence of the 3′-end overhang sequence, a complementary sequence of the cDNA sequence that is complementary to the RNA and formed by reverse transcription primed by the primer A.

In certain embodiments, the second region of the second bridging oligonucleotide is located at the 3′-end of the second bridging oligonucleotide.

In certain embodiments, the first region of the second bridging oligonucleotide is located at the 5′-end of the second bridging oligonucleotide.

In certain embodiments, the first bridging oligonucleotide does not comprise the third region, and/or, the second bridging oligonucleotide does not comprise the third region.

In certain embodiments, the second bridging oligonucleotide comprises a 5′ phosphate at the 5′-end.

In certain embodiments, the second bridging oligonucleotide comprises a free —OH at the 3′-end.

In certain embodiments, in step (3)(ii), the first bridging oligonucleotide is incapable of initiating an extension reaction (e.g., the 3′-end of the first bridging oligonucleotide is blocked), and/or, the first extension product obtained in step (2)(i) is incapable of initiating an extension reaction (e.g., the 3′-end of the first extension product obtained in step (2)(i) is blocked).

In certain embodiments, in step (2)(i)(a), the capture sequence A of the primer A is a random oligonucleotide sequence.

In some embodiments, the first extension product in step (2)(i)(b) comprises from 5′ to 3′: a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, the tag sequence B, a complementary sequence of the 3′-end overhang sequence, and a complementary sequence of the cDNA sequence that is complementary to the RNA and formed by reverse transcription primed by the primer A.

In certain embodiments, in step (2)(i)(a), the capture sequence A of the primer A is a poly(T) sequence or a specific sequence targeting a target nucleic acid.

In certain embodiments, the primer A also comprises a consensus sequence A, and optionally a tag sequence A, such as a random oligonucleotide sequence.

In certain embodiments, the capture sequence A is located at the 3′-end of the primer A.

In certain embodiments, the first extension product in step (2)(i)(b) comprises from 5′ to 3′: the consensus sequence A, optionally the tag sequence A, a cDNA sequence that is complementary to the RNA and formed by reverse transcription primed by the primer A, the 3′-end overhang sequence, a complementary sequence of the tag sequence B, and a complementary sequence of the consensus sequence B.

In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, the tag sequence B, a complementary sequence of the 3′-end overhang sequence, a complementary sequence of the cDNA sequence that is complementary to the RNA and formed by reverse transcription primed by the primer A, optionally a complementary sequence of the tag sequence A, and a complementary sequence of the consensus sequence A.

It is easy to understand that in step (3)(ii), after the first bridging oligonucleotide and the second bridging oligonucleotide are annealed to the oligonucleotide probe and the first nucleic acid molecule to be labeled which is at the corresponding position of the oligonucleotide probe, the ligation reaction for ligating the nucleic acid molecule hybridized with the first region and the nucleic acid molecule hybridized with the second region of the same first bridging oligonucleotide, and/or, ligating the nucleic acid molecule hybridized with the first region and the nucleic acid molecule hybridized with the second region of the same second bridging oligonucleotide, and the extension reaction in step (3)(ii) can be performed in any order, as long as a second nucleic acid molecule with a positioning tag can be obtained.

For example, when the ligation reaction and the extension reaction are performed in the same system, the second strand can be obtained by ligating the nucleic acid molecule hybridized with the first region and the nucleic acid molecule hybridized with the second region of the same first bridging oligonucleotide, and extending the second bridging oligonucleotide by an extension reaction. In this case, the polymerase used in the extension reaction preferably does not have strand displacement activity or 5′ to 3′ exonucleolytic activity.

For example, when the ligation reaction and the extension reaction are performed in different systems, the ligation reaction is performed followed by the extension reaction, the second strand can be obtained in the following exemplary manner:
(A) ligating the nucleic acid molecule hybridized with the first region and the nucleic acid molecule hybridized with the second region of the same first bridging oligonucleotide, and extending the second bridging oligonucleotide by an extension reaction to obtain the second strand; wherein, the polymerase used for the extension reaction preferably has or does not have strand displacement activity or 5′ to 3′ exonucleolytic activity;
or,
(B) ligating the nucleic acid molecule hybridized with the first region and the nucleic acid molecule hybridized with the second region of the same second bridging oligonucleotide, and extending the oligonucleotide probe by an extension reaction to obtain the second strand; wherein, the polymerase used for the extension reaction preferably has strand displacement activity or 5′ to 3′ exonucleolytic activity.

For example, when the ligation reaction and the extension reaction are performed in different systems, and the extension reaction is performed followed by the ligation reaction, the second strand can be obtained by extending the second bridging oligonucleotide by an extension reaction, and then ligating the nucleic acid molecule hybridized with the first region and the nucleic acid molecule hybridized with the second region of the same first bridging oligonucleotide. In this case, the polymerase used in the extension reaction preferably does not have strand displacement activity or 5′ to 3′ exonucleolytic activity.

An exemplary embodiment of the present application comprising step (1), step (2)(i) and step (3)(ii) is described in detail as follows:

I. An exemplary embodiment for preparing a cDNA strand using an RNA (e.g., mRNA) in a sample as a template comprises the following steps (as shown in FIG. 2):

(1) using a reverse transcriptase (e.g., a reverse transcriptase with terminal deoxynucleotidyl transferase activity) and a primer A to perform reverse transcription of the RNA molecule (e.g., mRNA molecule) in the permeabilized sample to generate a cDNA, and adding an overhang (e.g., an overhang comprising three cytosine nucleotides) to the 3′-end of the cDNA. Various reverse transcriptases with terminal deoxynucleotidyl transferase activity can be used to perform the reverse transcription reaction. In certain preferred embodiments, the reverse transcriptase used does not have RNase H activity.

In certain embodiments, the primer A comprises a poly(T) sequence and a consensus sequence A (CA). Normally, the poly(T) sequence is located at the 3′-end of the primer A to initiate the reverse transcription.

In certain embodiments, the primer A comprises a random oligonucleotide sequence that is capable of capturing an RNA without a poly(A) tail. Typically, the random oligonucleotide sequence is located at the 3′-end of the primer A to initiate the reverse transcription.

(2) using a primer B to anneal to or hybridize with the cDNA strand. The primer B comprises the consensus sequence B (CB), a unique molecular identifier (UMI) sequence and a complementary sequence of the 3′-end overhang of the cDNA. Subsequently, the nucleic acid fragment that hybridizes with or anneals to the primer B can be extended under the presence of a nucleic acid polymerase using the UMI sequence and the consensus sequence B as templates, thereby generating a nucleic acid molecule carrying a complementary sequence of the UMI sequence, and a complementary sequence of the consensus sequence B at the 3′-end.

Typically, the consensus sequence B is located upstream of the UMI sequence (e.g., located 5′ of the UMI sequence), and the complementary sequence of the 3′-end overhang of the cDNA strand is located at the 3′-end of the primer B.

For example, when the 3′-end of the cDNA strand comprises an overhang of three cytosine nucleotides, the primer B may comprise GGG at its 3′-end. In addition, the nucleotides of the primer B can also be modified (e.g., the primer B can be modified to comprise a locked nucleic acid) to enhance the binding affinity for the complementary pairing between the primer B and the 3′-end overhang of the cDNA strand.

Without being limited by any theory, various suitable nucleic acid polymerases (e.g., DNA polymerase or reverse transcriptase) can be used to perform the extension reaction, as long as they can use the sequence or partial sequence thereof of the primer B as a template to extend the annealed or hybridized nucleic acid fragment (reverse transcription product). In certain exemplary embodiments, the annealed or hybridized nucleic acid fragment (reverse transcription product) can be extended by using the same reverse transcriptase used in the aforementioned reverse transcription step.

In some embodiments, this step is performed simultaneously with step (1) (e.g., in the same reaction system).

In certain embodiments, the method optionally further comprises step (3): adding RNase H to digest the RNA strand in the RNA/cDNA hybrid to form a cDNA single strand.

In certain embodiments, the method does not comprise the step (3).

An exemplary structure of the cDNA strand prepared by the above exemplary embodiment comprises: the consensus sequence A, the cDNA sequence, the 3′-end overhang sequence, a complementary sequence of the UMI sequence, and a complementary sequence of the consensus sequence B.

II. An exemplary embodiment of labeling the 3′-end of a cDNA strand with a complementary sequence of an oligonucleotide probe (also known as chip sequence) to form a new nucleic acid molecule comprising the chip sequence information (i.e., a nucleic acid molecule labeled by the chip sequence) comprises the following steps (shown in FIG. 3):
providing a bridging oligonucleotide pair consisting of a first bridging oligonucleotide and a second bridging oligonucleotide, wherein the first bridging oligonucleotide and the second bridging oligonucleotide each independently comprise: a first region (P1) and a second region (P2), the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence B in the cDNA strand obtained in the above step I.

In certain embodiments, the first bridging oligonucleotide comprises an intermediate nucleotide sequence between the first region and the second region, such as an intermediate nucleotide sequence of 1 nt to 5 nt or 5 nt to 10 nt, that is, the first bridging oligonucleotide sequence comprises a third region located between the first region and the second region. In certain preferred embodiments, the first region and the second region in the first bridging oligonucleotide are adjacently connected without extra nucleotides between them, that is, the first bridging oligonucleotide sequence does not comprise a third region between the first region and the second region.

In certain embodiments, the second bridging oligonucleotide comprises an intermediate nucleotide sequence between the first region and the second region, such as an intermediate nucleotide sequence of 1 nt to 5 nt or 5 nt to 10 nt, that is, the second bridging oligonucleotide sequence comprises a third region located between the first region and the second region. In certain preferred embodiments, the first region and the second region in the second bridging oligonucleotide are adjacently connected without extra nucleotides between them, that is, the second bridging oligonucleotide sequence does not comprise a third region between the first region and the second region.

A new nucleic acid molecule comprising the chip sequence information (i.e., a nucleic acid molecule labeled by the chip sequence) can be obtained by: annealing or hybridizing the first bridging oligonucleotide and the second bridging oligonucleotide with the chip sequence and the cDNA strand obtained in step I above, and ligating the nucleic acid molecule hybridized with the first region and the nucleic acid molecule hybridized with the second region of the same first bridging oligonucleotide, and/or ligating the nucleic acid molecule hybridized with the first region and the nucleic acid molecule hybridized with the second region of the same second bridging oligonucleotide using a DNA ligase, and performing an extension reaction under the presence of a DNA polymerase. The ligation process and the extension reaction can be performed in any order.

An exemplary structure of the new nucleic acid molecule comprising the chip sequence information formed by the above exemplary embodiment comprises a nucleic acid strand and/or a complementary nucleic acid strand thereof, wherein the nucleic acid strand comprises from 5′ to 3′: the consensus sequence A, the cDNA sequence, the 3′-end overhang sequence, and a complementary sequence of the UMI sequence, a complementary sequence of the consensus sequence B, the first bridging oligonucleotide sequence, a complementary sequence of the tag sequence Y, and a complementary sequence of the consensus sequence X1.

In certain embodiments, the method comprises step (1), step (2)(ii), and step (3). In some embodiments, in step (2)(ii)(b), the first extension product comprises from 5′ to 3′: the consensus sequence A, a cDNA sequence that is complementary to the RNA and formed by reverse transcription primed by the primer A′, the 3′-end overhang sequence, optionally a complementary sequence of the tag sequence B, and a complementary sequence of the consensus sequence B.

In certain embodiments, in step (2)(ii)(c), the extension primer is the primer B′ as described above or a primer B″ or a random primer, wherein the primer B″ is capable of annealing to a complementary sequence or partial sequence thereof of the consensus sequence B, and capable of initiating an extension reaction.

In certain embodiments, in step (2)(ii)(c), the second extension product comprises from 5′ to 3′: a sequence that is complementary to the cDNA sequence and formed by an extension reaction primed by the extension primer, and a complementary sequence of the consensus sequence A.

Embodiments Comprising Step (1), Step (2)(ii) and Step (3)(i)

In certain embodiments, the method comprises step (1), step (2)(ii) and step (3)(i); wherein the consensus sequence X2 or partial sequence thereof is capable of annealing to a complementary sequence or partial sequence thereof of the consensus sequence A; the extension product obtained in step (3)(i) is the labeled nucleic acid molecule, which comprises: the first strand comprising the sequence of the first nucleic acid molecule to be labeled, and/or, the second strand comprising the sequence of the oligonucleotide probe.

It is easy to understand that the consensus sequence X2 in its entire nucleotide sequence is capable of annealing to a complementary sequence or partial fragment thereof of the consensus sequence A, and that the consensus sequence X2 in its partial nucleotide sequence is also capable of annealing to a complementary sequence or partial fragment thereof of the consensus sequence A.

In certain embodiments, the first strand comprises from 5′ to 3′: the sequence of the first nucleic acid molecule to be labeled, a complementary sequence of the tag sequence Y, and a complementary sequence of the consensus sequence X1.

In certain embodiments, the second strand comprises from 5′ to 3′: the consensus sequence X1, the tag sequence Y, the consensus sequence X2, and the cDNA sequence complementary to the sequence of the first nucleic acid molecule to be labeled.

Embodiments Comprising Step (1), Step (2)(ii) and Step (3)(i), for Generating the First Strand

In certain embodiments, the consensus sequence X2 or partial sequence thereof is capable of annealing to a complementary sequence or partial sequence thereof (e.g., 3′-end partial sequence thereof) of the consensus sequence A; the extension product obtained in step (3)(i) is the labeled nucleic acid molecule, which comprises the first strand comprising the sequence of the first nucleic acid molecule to be labeled.

In certain embodiments, in step (3)(i), the oligonucleotide probe is incapable of initiating an extension reaction (e.g., the 3′ end of the oligonucleotide probe is blocked).

In certain embodiments, in step (2)(ii)(a), the capture sequence A of the primer A′ is a random oligonucleotide sequence.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, and a complementary sequence of the consensus sequence A. In certain embodiments, the first strand comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the consensus sequence A, a complementary sequence of the tag sequence Y, and a complementary sequence of the consensus sequence X1.

In certain embodiments, in step (3), the first strand derived from each copy of the same kind of oligonucleotide probe has a different complementary sequence of the capture sequence A as a UMI.

In certain embodiments, in step (2)(ii)(a), the capture sequence A of the primer A′ is a poly(T) sequence or a specific sequence targeting a target nucleic acid.

In certain embodiments, the primer A′ also comprises a tag sequence A, such as a random oligonucleotide sequence.

In certain embodiments, the capture sequence A is located at the 3′-end of the primer A′.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, and a complementary sequence of the consensus sequence A. In certain embodiments, the first strand comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, a complementary sequence of the consensus sequence A, a complementary sequence of the tag sequence Y, and a complementary sequence of the consensus sequence X1.

In certain embodiments, in step (3), the first strand derived from each copy of the same kind of oligonucleotide probe has a different complementary sequence of the tag sequence A as a UMI.

Embodiments Comprising Step (1), Step (2)(ii) and Step (3)(i), for Generating the Second Strand

In some embodiments, the consensus sequence X2 or partial sequence thereof (e.g., 3′ end partial sequence thereof) is capable of annealing to a complementary sequence or partial sequence thereof of the consensus sequence A; and the extension product obtained in step (3)(i) is the labeled nucleic acid molecule, which comprises the second strand comprising the sequence of the oligonucleotide probe.

In certain embodiments, the second extension product obtained in step (2)(ii) is incapable of initiating an extension reaction (e.g., the 3′ end of the second extension product obtained in step (2)(ii) is blocked).

In certain embodiments, in step (2)(ii)(a), the capture sequence A of the primer A′ is a random oligonucleotide sequence.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, and a complementary sequence of the consensus sequence A. In certain embodiments, the second strand comprises from 5′ to 3′: the consensus sequence X1, the tag sequence Y, the consensus sequence X2, a cDNA sequence complementary to the first nucleic acid molecule to be labeled, the 3′-end overhang sequence, optionally a complementary sequence of the tag sequence B, and a complementary sequence of the consensus sequence B.

In certain embodiments, in step (3), the second strand derived from each copy of the same kind of oligonucleotide probe has a different capture sequence A as a UMI.

In certain embodiments, in step (2)(ii)(a), the capture sequence A of the primer A′ is a poly(T) sequence or a specific sequence targeting a target nucleic acid.

In certain embodiments, the primer A′ also comprises a tag sequence A, such as a random oligonucleotide sequence.

In certain embodiments, the capture sequence A is located at the 3′-end of the primer A′.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, and a complementary sequence of the consensus sequence A. In certain embodiments, the second strand comprises from 5′ to 3′: the consensus sequence X1, the tag sequence Y, the consensus sequence X2, the tag sequence A, a cDNA sequence complementary to the sequence of the first nucleic acid molecule to be labeled, the 3′-end overhang sequence, optionally a complementary sequence of the tag sequence B, and a complementary sequence of the consensus sequence B.

In certain embodiments, in step (3), the second strand derived from each copy of the same kind of oligonucleotide probe has a different tag sequence A as a UMI.

An exemplary embodiment of the present application comprising step (1), step (2)(ii) and step (3)(i) is described in detail as follows:

I. An exemplary embodiment for preparing a complementary strand of a cDNA strand comprising a complementary sequence of UMI close to the 3′-end by using an RNA (e.g., mRNA) in a sample as a template comprises the following steps (as shown in FIG. 5):

(1) using a reverse transcriptase (e.g., a reverse transcriptase with terminal deoxynucleotidyl transferase activity) and a primer A′ to perform reverse transcription of the RNA molecule (e.g., mRNA molecule) in the permeabilized sample to generate a cDNA, and added an overhang (e.g., an overhang comprising three cytosine nucleotides) to the 3′-end of the cDNA. Various reverse transcriptases with terminal deoxynucleotidyl transferase activity can be used to perform the reverse transcription reaction. In certain preferred embodiments, the reverse transcriptase used does not have RNase H activity.

In certain embodiments, the primer A′ comprises a poly(T) sequence, a UMI sequence, and a consensus sequence A (CA). Typically, the poly(T) sequence is located at the 3′-end of the primer A′ to initiate the reverse transcription, and the consensus sequence A is located upstream of the UMI sequence (e.g., located 5′ of the UMI sequence).

In certain embodiments, the primer A′ comprises a random oligonucleotide sequence and a consensus sequence A, and can be used to capture an RNA without a poly A tail. Typically, the random oligonucleotide sequence is located at the 3′-end of the primer A′ to initiate the reverse transcription.

(2) using a primer B′ to anneal to or hybridize with the cDNA strand. The primer B′ comprises a consensus sequence B (CB) and a complementary sequence of the 3′-end overhang of the cDNA. Extending the nucleic acid fragment hybridized or annealed with the primer B′ under the presence of a nucleic acid polymerase using the consensus sequence B as a template, to add a complementary sequence of the consensus sequence B (c(CB)) to the 3′-end of the cDNA strand, thereby generating a nucleic acid molecule carrying the complementary sequence of the consensus sequence B at the 3′-end.

Typically, the complementary sequence of the 3′-end overhang of the cDNA strand is located at the 3′-end of the primer B′.

For example, when the cDNA strand comprises an overhang of three cytosine nucleotides at the 3′-end, the primer B′ may comprise GGG at its 3′-end. In addition, the nucleotide of the primer B′ can also be modified (e.g., the primer B′ can be modified to comprise a locked nucleic acid) to enhance the binding affinity for the complementary pairing between the primer B′ and the 3′-end overhang of the cDNA strand.

Without being limited by any theory, various suitable nucleic acid polymerases (e.g., DNA polymerase or reverse transcriptase) can be used to perform the extension reaction, as long as they can extend the annealed or hybridized nucleic acid fragment (reverse transcription product) by using the sequence of the primer B′ or partial sequence thereof as a template. In certain exemplary embodiments, the annealed or hybridized nucleic acid fragment (reverse transcription product) can be extended using the same reverse transcriptase used in the aforementioned reverse transcription step.

In some embodiments, this step is performed simultaneously with step (1) (e.g., in the same reaction system).

In certain embodiments, the method optionally further comprises step (3): adding RNase H to digest the RNA strand in the RNA/cDNA hybrid to form a cDNA single strand.

In certain embodiments, the method does not comprise the step (3).

(4) using an extension primer to perform an extension reaction with the cDNA strand obtained in the previous step as a template to obtain an extension product; the extension primer is the primer B′ as described above, a random primer, or a primer B″, the primer B″ is capable of annealing to the consensus sequence B or partial sequence thereof, and capable of initiating the extension reaction.

An exemplary structure of the complementary strand of the cDNA strand prepared by the above exemplary embodiment comprises: the consensus sequence B, a complementary sequence of the 3′ overhang, a complementary sequence of the cDNA sequence, a complementary sequence of the UMI sequence, and a complementary sequence of the consensus sequence A.

II. An exemplary embodiment for labeling the 3′-end of a complementary strand of the cDNA strand with a complementary sequence of the oligonucleotide probe (also known as chip sequence) to form a new nucleic acid molecule comprising the chip sequence information (i.e., a nucleic acid molecule labeled by the chip sequence) comprises the following steps (shown in FIG. 7):

In some embodiments, the consensus sequence X2 or partial sequence thereof of the chip sequence is capable of annealing to a complementary sequence or partial sequence thereof of the consensus sequence A of the complementary strand of the cDNA strand obtained in the above step I. Thereby a new nucleic acid molecule comprising the chip sequence information (i.e., a nucleic acid molecule labeled by the chip sequence) can be obtained by annealing or hybridizing the complementary strand of the cDNA strand to the chip sequence, and performing an extension reaction under the presence of a polymerase.

An exemplary structure of the new nucleic acid molecule comprising the chip sequence information formed by the above exemplary embodiment comprises a nucleic acid strand and/or a complementary nucleic acid strand thereof, wherein the nucleic acid strand comprises from 5′ to 3′: the consensus sequence B, a complementary sequence of the 3′-end overhang, a complementary sequence of the cDNA sequence, a complementary sequence of the UMI sequence, a complementary sequence of the consensus sequence A, a complementary sequence of the tag sequence Y, and a complementary sequence of the consensus sequence X1.

Embodiments Comprising Step (1), Step (2)(ii) and Step (3)(ii)

In certain embodiments, the method comprises step (1), step (2)(ii) and step (3)(ii); wherein the second region of the second bridging oligonucleotide is capable of annealing to a complementary sequence or partial sequence thereof of the consensus sequence A of the second extension product obtained in step (2)(ii); the reaction product obtained in step (3)(ii) is the labeled nucleic acid molecule, which comprises: the first strand comprising the sequence of the first nucleic acid molecule to be labeled, and/or the second strand comprising the sequence of the oligonucleotide probe.

It is easy to understand that the second region of the second bridging oligonucleotide is capable of annealing to the complementary sequence or partial fragment thereof of the consensus sequence A of the second extension product obtained in step (2)(ii).

In certain embodiments, the first strand comprises from 5′ to 3′: the sequence of the first nucleic acid molecule to be labeled, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, and a cDNA sequence complementary to the sequence of the first nucleic acid molecule to be labeled.

Embodiments Comprising Step (1), Step (2)(ii) and Step (3)(ii), for Generating the First Strand

In certain embodiments, the second region of the second bridging oligonucleotide is capable of annealing to a complementary sequence or 3′-end partial sequence thereof of the consensus sequence A of the second extension product obtained in step (2)(ii), and the second region of the first bridging oligonucleotide has a free 3′ end.

In certain embodiments, the reaction product obtained in step (3)(ii) is the labeled nucleic acid molecule, which comprises the first strand.

In certain embodiments, the second region of the first bridging oligonucleotide is located at the 3′-end of the first bridging oligonucleotide.

In certain embodiments, the first region of the first bridging oligonucleotide is located at the 5′-end of the first bridging oligonucleotide. In certain embodiments, the first bridging oligonucleotide does not comprise the third region, and/or the second bridging oligonucleotide does not comprise the third region.

In certain embodiments, the first bridging oligonucleotide comprises a 5′ phosphate at the 5′-end.

In certain embodiments, the first bridging oligonucleotide comprises a free —OH at the 3′-end.

In certain embodiments, in step (3)(ii), the second bridging oligonucleotide is incapable of initiating an extension reaction (e.g., the 3′-end of the second bridging oligonucleotide is blocked), and/or, the oligonucleotide probe is incapable of initiating an extension reaction (e.g. the 3′-end of the oligonucleotide probe is blocked).

In certain embodiments, in step (2)(ii)(a), the capture sequence A of the primer A′ is a random oligonucleotide sequence.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, and a complementary sequence of the consensus sequence A. In certain embodiments, the first strand comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the consensus sequence A, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, in step (3), the first strand derived from each copy of the same kind of oligonucleotide probe has a different complementary sequence of the capture sequence A as a UMI.

In certain embodiments, in step (2)(ii)(a), the capture sequence A of the primer A′ is a poly(T) sequence or a specific sequence targeting a target nucleic acid.

In certain embodiments, the primer A′ also comprises a tag sequence A, such as a random oligonucleotide sequence.

In certain embodiments, the capture sequence A is located at the 3′-end of the primer A′.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, and a complementary sequence of the consensus sequence A. In certain embodiments, the first strand comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, a complementary sequence of the consensus sequence A, optionally a complementary sequence of the third region of the second bridging oligonucleotide, the first bridging oligonucleotide sequence, a complementary sequence of the tag sequence Y, and a complementary sequence of the consensus sequence X1.

In certain embodiments, in step (3), the first strand derived from each copy of the same kind of oligonucleotide probe has a different complementary sequence of the tag sequence A as a UMI.

It is easy to understand that in step (3)(ii), after the first bridging oligonucleotide and the second bridging oligonucleotide are annealed to the oligonucleotide probe and the first nucleic acid molecule to be labeled which is at corresponding position of the oligonucleotide probe, the ligation reaction for ligating the nucleic acid molecule hybridized with the first region and the nucleic acid molecule hybridized with the second region of the same first bridging oligonucleotide, and/or, ligating the nucleic acid molecule hybridized with the first region and the nucleic acid molecule hybridized with the second region of the same second bridging oligonucleotide and the extension reaction in step (3)(ii) can be performed in any order, as long as a second nucleic acid molecule carrying a positioning tag can be obtained.

For example, when the ligation reaction and the extension reaction are performed in the same system, the first strand can be obtained by ligating the nucleic acid molecule hybridized with the first region and the nucleic acid molecule hybridized with the second region of the same second bridging oligonucleotide, and extending the first bridging oligonucleotide by an extension reaction. In this case, the polymerase used in the extension reaction preferably does not have strand displacement activity or 5′ to 3′ exonucleolytic activity.

For example, when the ligation reaction and the extension reaction are performed in different systems, the ligation reaction is performed followed by the extension reaction, the first strand can be obtained in the following exemplary manner:
(A) ligating the nucleic acid molecule hybridized with the first region and the nucleic acid molecule hybridized with the second region of the same second bridging oligonucleotide, and extending the first bridging oligonucleotide by an extension reaction to obtain the first strand; wherein, the polymerase used for the extension reaction preferably has or does not have strand displacement activity or 5′ to 3′ exonucleolytic activity;
or,
(B) ligating the nucleic acid molecule hybridized with the first region and the nucleic acid molecule hybridized with the second region of the same first bridging oligonucleotide, and extending the first nucleic acid molecule to be labeled by an extension reaction to obtain the first strand; wherein, the polymerase used for the extension reaction preferably has strand displacement activity or 5′ to 3′ exonucleolytic activity.

For example, when the ligation reaction and the extension reaction are performed in different systems, and the extension reaction is performed followed by the ligation reaction, the first strand can be obtained by extending the first bridging oligonucleotide by an extension reaction, and then ligating the nucleic acid molecule hybridized with the first region and the nucleic acid molecule hybridized with the second region of the same second bridging oligonucleotide. In this case, the polymerase used in the extension reaction preferably does not have strand displacement activity or 5′ to 3′ exonucleolytic activity.

Embodiments Comprising Step (1), Step (2)(ii) and Step (3)(ii), for Generating the Second Strand

In certain embodiments, the second region of the second bridging oligonucleotide is capable of annealing to a complementary sequence or partial sequence thereof of the consensus sequence A of the second extension product obtained in step (2)(ii), and the second region of the second bridging oligonucleotide has a free 3′ end.

In certain embodiments, the reaction product obtained in step (3)(ii) is the labeled nucleic acid molecule, which comprises the second strand.

In certain embodiments, the second region of the second bridging oligonucleotide is located at the 3′-end of the second bridging oligonucleotide.

In certain embodiments, the first region of the second bridging oligonucleotide is located at the 5′-end of the second bridging oligonucleotide.

In certain embodiments, the first bridging oligonucleotide does not comprise the third region, and/or the second bridging oligonucleotide does not comprise the third region.

In certain embodiments, the second bridging oligonucleotide comprises a 5′ phosphate at the 5′-end.

In certain embodiments, the second bridging oligonucleotide comprises a free —OH at the 3′-end.

In certain embodiments, in step (3)(ii), the first bridging oligonucleotide is incapable of initiating an extension reaction (e.g., the 3′-end of the first bridging oligonucleotide is blocked), and/or, the second extension product obtained in step (2)(ii) is incapable of initiating an extension reaction (e.g., the 3′-end of the second extension product obtained in step (2)(ii) is blocked).

In certain embodiments, in step (2)(ii)(a), the capture sequence A of the primer A′ is a random oligonucleotide sequence.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, and a complementary sequence of the consensus sequence A. In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, a cDNA sequence complementary to the sequence of the first nucleic acid molecule to be labeled, the 3′-end overhang sequence, optionally a complementary sequence of the tag sequence B, and a complementary sequence of the consensus sequence B.

In certain embodiments, in step (3), the second strand derived from each copy of the same kind of oligonucleotide probe has a different capture sequence A as a UMI.

In certain embodiments, in step (2)(ii)(a), the capture sequence A of the primer A′ is a poly(T) sequence or a specific sequence targeting a target nucleic acid.

In certain embodiments, the primer A′ also comprises a tag sequence A, such as a random oligonucleotide sequence.

In certain embodiments, the capture sequence A is located at the 3′-end of the primer A′.

In certain embodiments, in step (2)(ii)(c), the extension primer is the primer B′. In certain embodiments, in step (2)(ii)(c), the second extension product comprises from 5′ to 3′: the consensus sequence B, optionally the tag sequence B, a complementary sequence of the 3′-end overhang sequence, a complementary sequence of the cDNA sequence that is complementary to the RNA and formed by reverse transcription primed by the primer A′, a complementary sequence of the tag sequence A, and a complementary sequence of the consensus sequence A. In certain embodiments, the second strand comprises from 5′ to 3′: the consensus sequence X1, the tag sequence Y, the consensus sequence X2, optionally a complementary sequence of the third region of the first bridging oligonucleotide, the second bridging oligonucleotide sequence, the tag sequence A, a cDNA sequence complementary to the sequence of the first nucleic acid molecule to be labeled, the 3′-end overhang sequence, optionally a complementary sequence of the tag sequence B, and a complementary sequence of the consensus sequence B.

In certain embodiments, in step (3), the second strand derived from each copy of the same kind of oligonucleotide probe has a different tag sequence A as a UMI.

It is easy to understand that in step (3)(ii), after the first bridging oligonucleotide and the second bridging oligonucleotide are annealed to the oligonucleotide probe and the first nucleic acid molecule to be labeled which is at the corresponding position of the oligonucleotide probe, the ligation reaction for ligating the nucleic acid molecule hybridized with the first region and the nucleic acid molecule hybridized with the second region of the same first bridging oligonucleotide, and/or, ligating the nucleic acid molecule hybridized with the first region and the nucleic acid molecule hybridized with the second region of the same second bridging oligonucleotide, and the extension reaction in step (3)(ii) can be performed in any order, as long as a second nucleic acid molecule carrying a positioning tag can be obtained.

For example, when the ligation reaction and the extension reaction are performed in the same system, the second strand can be obtained by ligating the nucleic acid molecule hybridized with the first region and the nucleic acid molecule hybridized with the second region of the same first bridging oligonucleotide, and extending the second bridging oligonucleotide by an extension reaction. In this case, the polymerase used in the extension reaction preferably does not have strand displacement activity or 5′ to 3′ exonucleolytic activity.

For example, when the ligation reaction and the extension reaction are performed in different systems, the ligation reaction is performed followed by the extension reaction, the second strand can be obtained in the following exemplary manner:
(A) ligating the nucleic acid molecule hybridized with the first region and the nucleic acid molecule hybridized with the second region of the same first bridging oligonucleotide, and extending the second bridging oligonucleotide by an extension reaction to obtain the second strand; wherein, the polymerase used for the extension reaction preferably has or does not have strand displacement activity or 5′ to 3′ exonucleolytic activity;
or,
(B) ligating the nucleic acid molecule hybridized with the first region and the nucleic acid molecule hybridized with the second region of the same second bridging oligonucleotide, and extending the oligonucleotide probe by an extension reaction to obtain the second strand; wherein, the polymerase used for the extension reaction preferably has strand displacement activity or 5′ to 3′ exonucleolytic activity.

For example, when the ligation reaction and the extension reaction are performed in different systems, and the extension reaction is performed followed by the ligation reaction, the second strand can be obtained by extending the second bridging oligonucleotide by an extension reaction, and then ligating the nucleic acid molecule hybridized with the first region and the nucleic acid molecule hybridized with the second region of the same first bridging oligonucleotide. In this case, the polymerase used in the extension reaction preferably does not have strand displacement activity or 5′ to 3′ exonucleolytic activity.

An exemplary embodiment of the present application comprising step (1), step (2)(ii) and step (3)(ii) is described in detail as follows:

I. An exemplary embodiment for preparing a complementary strand of a cDNA strand by using an RNA (e.g., mRNA) in a sample as a template comprises the following steps (as shown in FIG. 5):

(1) using a reverse transcriptase (e.g., a reverse transcriptase with terminal deoxynucleotidyl transferase activity) and a primer A′ to perform reverse transcription of an RNA molecule (e.g., mRNA molecule) in the permeabilized sample to generate a cDNA, and adding an overhang (e.g., an overhang comprising three cytosine nucleotides) to the 3′-end of the cDNA. Various reverse transcriptases with terminal deoxynucleotidyl transferase activity can be used to perform the reverse transcription reaction. In certain preferred embodiments, the reverse transcriptase used does not have RNase H activity.

In certain embodiments, the primer A′ comprises a poly(T) sequence, a UMI sequence, and a consensus sequence A (CA). Typically, the poly(T) sequence is located at the 3′-end of the primer A′ to initiate the reverse transcription, and the consensus sequence A is located upstream of the UMI sequence (e.g., located 5′ of the UMI sequence).

In certain embodiments, the primer A′ comprises a random oligonucleotide sequence and a consensus sequence A, and can be used to capture an RNA without a poly A tail. Typically, the random oligonucleotide sequence is located at the 3′-end of the primer A′ to initiate the reverse transcription.

(2) using a primer B′ to anneal to or hybridize with the cDNA strand. The primer B′ comprises a consensus sequence B (CB) and a complementary sequence of the 3′-end overhang of the cDNA. Extending the nucleic acid fragment hybridized or annealed with the primer B′ under the presence of a nucleic acid polymerase using the consensus sequence B as a template, to add a complementary sequence of the consensus sequence B (c(CB)) to the 3′-end of the cDNA strand, thereby generating a nucleic acid molecule carrying the complementary sequence of the consensus sequence B at the 3′-end.

Typically, the complementary sequence of the 3′-end overhang of the cDNA strand is located at the 3′-end of the primer B′.

For example, when the cDNA strand comprises an overhang of three cytosine nucleotides at the 3′-end, the primer B′ may comprise GGG at its 3′-end. In addition, the nucleotides of the primer B′ can also be modified (e.g., the primer B′ can be modified to comprise a locked nucleic acid) to enhance the binding affinity for the complementary pairing between the primer B′ and the 3′-end overhang of the cDNA strand.

Without being limited by any theory, various suitable nucleic acid polymerases (e.g., DNA polymerase or reverse transcriptase) can be used to perform the extension reaction, as long as they can extend the annealed or hybridized nucleic acid fragment (reverse transcription product) by using the sequence of the primer B′ or partial sequence thereof as a template. In certain exemplary embodiments, the annealed or hybridized nucleic acid fragment (reverse transcription product) can be extended by using the same reverse transcriptase used in the aforementioned reverse transcription step.

In some embodiments, this step is performed simultaneously with step (1) (e.g., in the same reaction system).

In certain embodiments, the method optionally further comprises step (3): adding RNase H to digest the RNA strand in the RNA/cDNA hybrid to form a cDNA single strand.

In certain embodiments, the method does not comprise the step (3).

(4) using an extension primer to perform an extension reaction with the cDNA strand obtained in the previous step as a template to obtain an extension product; the extension primer is the primer B′ as described above, a random primer, or a primer B″, the primer B″ is capable of annealing to the consensus sequence B or partial sequence thereof, and capable of initiating the extension reaction.

An exemplary structure of the complementary strand of the cDNA strand prepared by the above exemplary embodiment comprises: the consensus sequence B, a complementary sequence of the 3′-end overhang, a complementary sequence of the cDNA sequence, a complementary sequence of the UMI sequence, and a complementary sequence of the consensus sequence A.

II. An exemplary embodiment for labeling the 3′-end of a complementary strand of a cDNA strand with a complementary sequence of an oligonucleotide probe (also known as chip sequence) to form a new nucleic acid molecule comprising the chip sequence information (i.e., a nucleic acid molecule labeled by the chip sequence) comprises the following steps (shown in FIG. 6):
providing a bridging oligonucleotide pair consisting of a first bridging oligonucleotide and a second bridging oligonucleotide, wherein the first bridging oligonucleotide and the second bridging oligonucleotide each independently comprise: a first region (P1) and a second region (P2), the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence A in the complementary strand of the cDNA strand obtained in the above step I.

In certain embodiments, the first bridging oligonucleotide comprises an intermediate nucleotide sequence between the first region and the second region, such as an intermediate nucleotide sequence of 1-5 nt or 5-10 nt, that is, the first bridging oligonucleotide sequence comprises a third region located between the first region and the second region. In certain preferred embodiments, the first region and the second region in the first bridging oligonucleotide are adjacently connected without extra nucleotides between them, that is, the first bridging oligonucleotide sequence does not comprise a third region between the first region and the second region.

In certain embodiments, the second bridging oligonucleotide comprises an intermediate nucleotide sequence between the first region and the second region, such as an intermediate nucleotide sequence of 1-5 nt or 5-10 nt, that is, the second bridging oligonucleotide sequence comprises a third region located between the first region and the second region. In certain preferred embodiments, the first region and the second region in the second bridging oligonucleotide are adjacently connected without extra nucleotides between them, that is, the second bridging oligonucleotide sequence does not comprise a third region between the first region and the second region.

A new nucleic acid molecule comprising the chip sequence information (i.e., a nucleic acid molecule labeled by the chip sequence) can be obtained by: annealing or hybridizing the first bridging oligonucleotide and the second bridging oligonucleotide with the chip sequence and the complementary strand of the cDNA strand obtained in the above step I, and ligating the nucleic acid molecule hybridized with the first region and the nucleic acid molecule hybridized with the second region of the same first bridging oligonucleotide, and/or, ligating the nucleic acid molecule hybridized with the first region and the nucleic acid molecule hybridized with the second region of the same second bridging oligonucleotide using a DNA ligase, and performing an extension reaction under the presence of a DNA polymerase. The ligation process and the extension reaction can be performed in any order.

An exemplary structure of the new nucleic acid molecule comprising the chip sequence information formed by the above exemplary embodiment comprises a nucleic acid strand and/or a complementary nucleic acid strand thereof, wherein the nucleic acid strand comprises from 5′ to 3′: the consensus sequence B, a complementary sequence of the 3′-end overhang, a complementary sequence of the cDNA sequence, a complementary sequence of the UMI sequence, a complementary sequence of the consensus sequence A, the first bridging oligonucleotide sequence, a complementary sequence of the tag sequence Y, and a complementary sequence of the consensus sequence X1.

In certain embodiments, in step (2)(i)(b) of the method, the cDNA strand anneals to the primer B through its 3′-end overhang, and, under the presence of a nucleic acid polymerase (e.g., DNA polymerase or reverse transcriptase), the cDNA strand is extended using the primer B as a template to generate the first extension product.

In certain embodiments, in step (2)(ii)(b) of the method, the cDNA strand anneals to the primer B′ through its 3′-end overhang, and, under the presence of a nucleic acid polymerase (e.g., DNA polymerase or reverse transcriptase), the cDNA strand is extended using the primer B′ as a template to generate the first extension product.

In certain embodiments, the 3′-end overhang has a length of at least 1, at least 2, at least 3, at least 4, at least 5, at least 6, at least 7, at least 8, at least 9, at least 10 or more nucleotides. In certain embodiments, the 3′-end overhang is a 3′-end overhang of 2-5 cytosine nucleotides (e.g., a CCC overhang).

In some embodiments, in step (2), the biological sample is permeabilized before performing the preprocessing.

In certain embodiments, the biological sample is a tissue sample.

In certain embodiments, the tissue sample is a tissue section.

In certain embodiments, the tissue section is prepared from a fixed tissue, for example, a formalin-fixed paraffin-embedded (FFPE) tissue or a deep-frozen tissue.

In certain embodiments, when the biological sample is in contact with the nucleic acid array, each cell of the biological sample individually occupies one or more microdots in the nucleic acid array (i.e., each cell is individually contacted with one or more microdots in the nucleic acid array).

In certain embodiments, the reverse transcription in step (2) is performed by using a reverse transcriptase.

In certain embodiments, the reverse transcriptase has terminal deoxynucleotidyl transferase activity.

In certain embodiments, the reverse transcriptase is capable of synthesizing a cDNA strand using an RNA (e.g., mRNA) as a template, and adding an overhang at the 3′-end of the cDNA strand.

In certain embodiments, the reverse transcriptase is capable of adding to the 3′-end of the cDNA strand an overhang with a length of at least 1, at least 2, at least 3, at least 4, at least 5, at least 6, at least 7, at least 8, at least 9, at least 10 or more nucleotides.

In certain embodiments, the reverse transcriptase is capable of adding to the 3′-end of the cDNA strand an overhang of 2-5 cytosine nucleotides (e.g., a CCC overhang).

In certain embodiments, the reverse transcriptase is selected from the group consisting of M-MLV reverse transcriptase, HIV-1 reverse transcriptase, AMV reverse transcriptase, telomerase reverse transcriptase, as well as variants, modified products and derivatives thereof having the reverse transcriptase activity of the above-mentioned reverse transcriptases.

In certain embodiments, steps (2) and (3) have one or more characteristics selected from the following:
(1) the primer A, primer A′, primer B, primer B′, the first bridging oligonucleotide, and the second bridging oligonucleotide each independently comprise or consist of natural nucleotides (e.g., deoxyribonucleotides or ribonucleotides), modified nucleotides, non-natural nucleotides, or any combination thereof; in certain embodiments, the primer A and primer A′ can initiate an extension reaction;
(2) the primer B comprises a modified nucleotide (e.g., locked nucleic acid); in certain embodiments, the primer B comprises one or more modified nucleotides (e.g., one or more locked nucleic acids) at the 3′-end;
(3) the primer B′ comprises a modified nucleotide (e.g., locked nucleic acid); in certain embodiments, the primer B′ comprises one or more modified nucleotides (e.g., one or more locked nucleic acids) at the 3′-end;
(4) the tag sequence A and tag sequence B each independently have a length of 5 to 200 nt (e.g., 5 to 30 nt, 6 to 15 nt);
(5) the consensus sequence A and consensus sequence B each independently have a length of 10 to 200 nt (e.g., 10 to 100 nt, 20 to 100 nt, 25 to 100 nt, 5 to 10 nt, 10 to 15 nt, 15 to 20 nt, 20 to 50 nt, 20 to 30 nt, 30 to 40 nt, 40 to 50 nt, 50 to 100 nt);
(6) the primer A, primer A′, primer B, and primer B′ each independently have a length of 4 to 200 nt (e.g., 5 to 200 nt, 15 to 230 nt, 26 to 115 nt, 10 to 130 nt, 10 to 20 nt, 20 to 50 nt, 20 to 30 nt, 30 to 40 nt, 40 to 50 nt, 50 to 100 nt, 100 to 150 nt, 150 to 200 nt);
(7) the first region and the second region of the first bridging oligonucleotide each independently have a length of 3 to 100 nt (e.g., 20 to 100 nt, 3 to 10 nt, 10 to 15 nt, 15 to 20 nt, 20 to 70 nt, 20 to 30 nt, 30 to 40 nt, 40 to 50 nt, 50 to 100 nt);
(8) the first region and the second region of the second bridging oligonucleotide each independently have a length of 3 to 100 nt (e.g., 20 to 100 nt, 3 to 10 nt, 10 to 15 nt, 15 to 20 nt, 20 to 70 nt, 20 to 30 nt, 30 to 40 nt, 40 to 50 nt, 50 to 100 nt);
(9) the third region of the first bridging oligonucleotide and the third region of the second bridging oligonucleotide each independently have a length of 0 to 50 nt (e.g., 0 nt, 0 to 10 nt, 10 to 15 nt, 15 to 20 nt, 20 to 30 nt, 30 to 40 nt, 40 to 50 nt);
(10) the first bridging oligonucleotide and the second bridging oligonucleotide each independently have a length of 6 to 200 nt (e.g., 20 to 100 nt, 20 to 70 nt, 6 to 15 nt, 15 to 20 nt, 20 to 30 nt, 30 to 40 nt, 40 to 50 nt, 50 to 100 nt, 100 to 150 nt, 150 to 200 nt);
(11) the poly(T) sequence comprises at least 5, or at least 20 (e.g., 6 to 100, 10 to 50) deoxythymidine nucleoside residues;
(12) the random oligonucleotide sequence has a length of 5 to 200 (e.g., 5 nt, 5 to 30 nt, 6 to 15 nt).

In certain embodiments, the method further comprises: (4) recovering and purifying the second nucleic acid molecule population.

In certain embodiments, the obtained second nucleic acid molecule population and/or complements thereof are used for constructing a transcriptome library or for transcriptome sequencing.

In certain embodiments, the oligonucleotide probe in step (1) has one or more characteristics selected from the following:
(1) the consensus sequence X1, tag sequence Y, and consensus sequence X2 each independently comprise or consist of natural nucleotides (e.g., deoxyribonucleotides or ribonucleotides), modified nucleotides, non-natural nucleotides (e.g., peptide nucleic acid (PNA) or locked nucleic acid), or any combination thereof;
(2) the consensus sequence X1, tag sequence Y, and consensus sequence X2 each independently have a length of 2 to 200 nt (e.g., 10 to 200 nt, 25 to 100 nt, 10 to 30 nt, 10 to 100 nt, 5 to 10 nt, 10 to 15 nt, 15 to 20 nt, 20-30 nt, 30-40 nt, 40-50 nt, 50-100 nt).

In certain embodiments, the oligonucleotide probe is coupled to the solid support via a linker.

In certain embodiments, the linker is a linking group capable of coupling with an activating group, and the surface of the solid support is modified with the activating group.

In certain embodiments, the linker comprises —SH, -DBCO, or —NHS.

In certain embodiments, the linker is -DBCO, and the surface of the solid support is

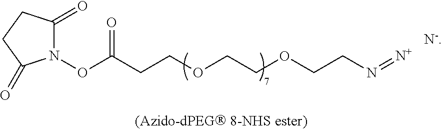

In some embodiments, the nucleic acid array of step (1) has one or more characteristics selected from the following:
(1) the oligonucleotide probes coupled to the same solid support have the same consensus sequence X1 and/or the same consensus sequence X2;
(2) the consensus sequence X1 of the oligonucleotide probe comprises a cleavage site; in some embodiments, the cleavage site can be cleaved or broken by means selected from nicking enzyme digestion, USER enzyme digestion, light-responsive excision, chemical excision, or CRISPR-mediated excision.

In some embodiments, the nucleic acid array of step (1) is provided by the following steps:
(1) providing multiple kinds of carrier sequences, each kind of carrier sequence comprises at least one copy (e.g., multiple copies) of the carrier sequence, and the carrier sequence comprises in the direction from 5′ to 3′: a complementary sequence of the consensus sequence X2, a complementary sequence of the tag sequence Y, and an immobilization sequence; wherein, the complementary sequence of the tag sequence Y of each kind of carrier sequence is different from one another;
(2) attaching the multiple kinds of carrier sequences to the surface of a solid support (e.g., a chip);
(3) providing an immobilization primer, and using the carrier sequence as a template to perform a primer extension reaction to generate an extension product, so as to obtain the oligonucleotide probe; wherein the immobilization primer comprises the sequence of the consensus sequence X1, and is capable of annealing to the immobilization sequence of the carrier sequence and initiating an extension reaction; in some embodiments, the extension product in the direction from 5′ to 3′ comprises or consists of: the consensus sequence X1, the tag sequence Y and the consensus sequence X2;
(4) linking the immobilization primer to the surface of the solid support; wherein steps (3) and (4) are performed in any order;
(5) optionally, the immobilization sequence of the carrier sequence further comprises a cleavage site, and the cleavage can be selected from nicking enzyme digestion, USER enzyme digestion, light-responsive excision, chemical excision or CRISPR-mediated excision; performing cleavage at the cleavage site comprised in the immobilization sequence of the carrier sequence to digest the carrier sequence, so as to separate the extension product in step (3) from the template (i.e., the carrier sequence) from which the extension product is generated, thereby linking the oligonucleotide probe to the surface of the solid support (e.g., chip). In certain embodiments, the method further comprises separating the extension product in step (3) from the template (i.e., carrier sequence) from which the extension product is generated through high-temperature denaturation.

In certain embodiments, each kind of carrier sequence is a DNB formed from a concatemer of multiple copies of the carrier sequence.

In certain embodiments, the multiple kinds of carrier sequences are provided in step (1) by the following steps:
(i) providing multiple kinds of carrier-template sequences, each carrier-template sequence comprises a complementary sequence of a carrier sequence;
(ii) using each kind of carrier-template sequence as a template to perform a nucleic acid amplification reaction so as to obtain an amplification product of each kind of carrier-template sequence, wherein the amplification product comprises at least one copy of the carrier sequence; in certain embodiments, rolling circle replication is performed to obtain a DNB formed from a concatemer of the carrier sequence.

In some embodiments, the solid support in step (1) has one or more characteristics selected from the following:
(1) the solid support is selected from the group consisting of latex bead, dextran bead, polystyrene surface, polypropylene surface, polyacrylamide gel, gold surface, glass surface, chip, sensor, electrode and silicon wafer; in some embodiments, the solid support is a chip;
(2) the solid support is planar, spherical or porous;
(3) the solid support can be used as a sequencing platform, such as a sequencing chip; in some embodiments, the solid support is a sequencing chip for Illumina, MGI or Thermo Fisher sequencing platform; and
(4) the solid support is capable of releasing the oligonucleotide probe spontaneously or upon exposure to one or more stimuli (e.g., temperature change, pH change, exposure to specific chemicals or phases, exposure to light, exposure to reducing agents, etc.).

### Methods for Constructing a Library of Nucleic Acid Molecules

In another aspect, the present application also provides a method for constructing a library of nucleic acid molecules, which comprises,
(a) generating a population of labeled nucleic acid molecules according to the method as described above;
(b) randomly fragmenting the nucleic acid molecules in the population of labeled nucleic acid molecules and linking an adapter thereto; and
(c) optionally, amplifying and/or enriching the product of step (b);
thereby obtaining the library of nucleic acid molecules.

In certain embodiments, the library of nucleic acid molecules is used for sequencing, such as transcriptome sequencing, such as single-cell transcriptome sequencing (e.g., 5′ or 3′ transcriptome sequencing).

In certain embodiments, before performing step (b), the method further comprises a step (pre-b): amplifying and/or enriching the population of labeled nucleic acid molecules.

In certain embodiments, in step (pre-b), the population of labeled nucleic acid molecules is subjected to a nucleic acid amplification reaction to generate an amplification product.

In certain embodiments, the amplification reaction is performed using at least a primer C and/or a primer D, wherein the primer C is capable of hybridizing with or annealing to a complementary sequence or partial sequence thereof of the consensus sequence X1, and initiating an extension reaction; the primer D is capable of hybridizing with or annealing to the nucleic acid molecule comprising the tag sequence Y in the population of labeled nucleic acid molecules, and initiating an extension reaction.

In certain embodiments, the nucleic acid amplification reaction in step (pre-b) is performed by using a nucleic acid polymerase (e.g., a DNA polymerase, such as a DNA polymerase with strand displacement activity and/or high fidelity).

In certain embodiments, in step (b) of the method, the nucleic acid molecules are randomly fragmented and linked with an adapter by using a transposase.

In some embodiments, in step (b) of the method, by using a transposase, the nucleic acid molecules obtained in the previous step are randomly fragmented and the resulting fragments are linked with a first adapter and a second adapter at both ends, respectively.

In certain embodiments, the transposase is selected from the group consisting of Tn5 transposase, MuA transposase, Sleeping Beauty transposase, Mariner transposase, Tn7 transposase, Tn10 transposase, Ty1 transposase, Tn552 transposase, as well as variants, modified products and derivatives thereof having the transposase activity of the above-mentioned transposases.

In certain embodiments, the transposase is a Tn5 transposase.

In some embodiments, in step (c), the product of step (b) is amplified using at least a primer C′ and/or a primer D′, wherein the primer C′ is capable of hybridizing with or annealing to the first adapter, and initiating an extension reaction, and the primer D′ is capable of hybridizing with or annealing to the second adapter, and initiating an extension reaction.

In some embodiments, in step (c), at least the primer C as described above and/or a primer D′ is used to amplify the product of step (b); wherein the primer D′ is capable of hybridizing with or annealing to the first adapter or the second adapter, and initiating an extension reaction.

### Methods for Performing Transcriptome Sequencing

In another aspect, the present application also provides a method for transcriptome sequencing of a cell in a sample, which comprises:
(1) constructing a library of nucleic acid molecules according to the method as described above; and
(2) sequencing the library of nucleic acid molecules.

### Kit

In another aspect, the application also provides a kit, which comprises:
(i) a nucleic acid array for labeling nucleic acids, which comprises a solid support, in which the solid support is coupled with multiple kinds of oligonucleotide probes; each kind of oligonucleotide probe comprises at least one copy; and, the oligonucleotide probe in the direction from 5′ to 3′ comprises or consists of: a consensus sequence X1, a tag sequence Y, and a consensus sequence X2, wherein,
each kind of oligonucleotide probe has a different tag sequence Y, and the tag sequence Y has a nucleotide sequence unique to the position of the oligonucleotide probe on the solid support;
(ii) a primer set comprising a primer A and a primer B or comprising a primer A′ and a primer B′, wherein:
the primer A comprises a capture sequence A, in which the capture sequence A is capable of annealing to an RNA (e.g., mRNA) to be captured and initiating an extension reaction;
the primer B comprises a consensus sequence B, a complementary sequence of a 3′-end overhang, and optionally a tag sequence B; in certain embodiments, the complementary sequence of a 3′-end overhang is located at the 3′-end of the primer B; in certain embodiments, the consensus sequence B is located upstream of the complementary sequence of a 3′-end overhang (e.g., located at the 5′-end of the primer B); wherein the 3′-end overhang refers to one or more non-templated nucleotides comprised in the 3′-end of a cDNA strand generated by reverse transcription using an RNA captured by the capture sequence A of the primer A as a template;
the primer A′ comprises a consensus sequence A and a capture sequence A; in some embodiments, the capture sequence A is located at the 3′-end of the primer A′; in some embodiments, the consensus sequence A is located upstream of the capture sequence A (e.g., located at the 5′-end of the primer A′);
the primer B′ comprises a consensus sequence B, a complementary sequence of a 3′-end overhang, and optionally a tag sequence B; in certain embodiments, the complementary sequence of a 3′-end overhang is located at the 3′-end of the primer B′; in certain embodiments, the consensus sequence B is located upstream of the complementary sequence of a 3′-end overhang (e.g., located at the 5′-end of the primer B′); wherein the 3′-end overhang refers to one or more non-templated nucleotides comprised in the 3′-end of a cDNA strand generated by reverse transcription using an RNA captured by the capture sequence A of the primer A′ as a template.

In certain embodiments, each oligonucleotide probe comprises one copy.

In certain embodiments, each oligonucleotide probe comprises multiple copies.

In certain embodiments, a region with one kind of oligonucleotide probe coupled to the solid support is referred to as a microdot. It is easy to understand that when each kind of oligonucleotide probe comprises one copy, each microdot is coupled with one oligonucleotide probe, and the oligonucleotide probes of different microdots have different tag sequences Y; when each kind of oligonucleotide nucleotide probe comprises multiple copies, each microdot is coupled with multiple oligonucleotide probes of the same kind, the oligonucleotide probes in the same microdot have the same tag sequence Y, and the oligonucleotide probes in different microdots have different tag sequences Y.

In certain embodiments, the solid support comprises a plurality of microdots, each microdot is coupled with one kind of oligonucleotide probe, and each kind of oligonucleotide probe may comprise one or more copies.

In certain embodiments, the solid support comprises a plurality of (e.g., at least 10, at least 10², at least 10³, at least 10⁴, at least 10⁵, at least 10⁶, at least 10⁷, at least 10⁸, or more) microdots. In certain embodiments, the solid support comprises at least 10⁴ (e.g., at least 10⁴, at least 10⁵, at least 10⁶, at least 10⁷, at least 10⁸, at least 10⁹, at least 10¹⁰, at least 10¹¹, or at least 10¹²) microdots/mm².

In some embodiments, the spacing between adjacent microdots is less than 100 μm, less than 50 μm, less than 10 μm, less than 5 μm, less than 1 μm, less than 0.5 μm, less than 0.1 μm, less than 0.05 μm, or less than 0.01 μm.

In certain embodiments, the microdot has a size (e.g., equivalent diameter) of less than 100 μm, less than 50 μm, less than 10 μm, less than 5 μm, less than 1 μm, less than 0.5 μm, less than 0.1 μm, less than 0.05 μm, or less than 0.01 μm.

In certain embodiments, the kit comprises: the nucleic acid array for labeling nucleic acids as described in (i), the primer set comprising the primer A and primer B as described in (ii), and, (iii) a first bridging oligonucleotide and a second bridging oligonucleotide; wherein the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence B of the primer B.

In certain embodiments, the capture sequence A of the primer A is a random oligonucleotide sequence.

In certain embodiments, the capture sequence A of the primer A is a poly(T) sequence or a sequence specific for a target nucleic acid. In certain embodiments, the primer A further comprises a consensus sequence A and optionally a tag sequence A, such as a random oligonucleotide sequence. In certain embodiments, the capture sequence A is located at the 3′-end of the primer A, and the consensus sequence A is located upstream (e.g., located at the 5′-end) of the primer A.

In certain embodiments, the primer B comprises a consensus sequence B, a complementary sequence of the 3′-end overhang, and a tag sequence B.

In certain embodiments, the primer B comprises a modified nucleotide (e.g., locked nucleic acid). In certain embodiments, the primer B comprises one or more modified nucleotides (e.g., one or more locked nucleic acids) at the 3′-end.

In certain embodiments, the second region of the second bridging oligonucleotide is capable of annealing to a complementary sequence or partial sequence thereof (e.g., the 3′ end partial sequence thereof) of the consensus sequence B of the primer B.

In certain embodiments, the second region of the first bridging oligonucleotide is located at the 3′-end of the first bridging oligonucleotide.

In certain embodiments, the first region of the first bridging oligonucleotide is located at the 5′-end of the first bridging oligonucleotide.

In certain embodiments, the first bridging oligonucleotide comprises a 5′ phosphate at the 5′-end.

In certain embodiments, the first bridging oligonucleotide comprises a free —OH at the 3′-end.

In certain embodiments, the second bridging oligonucleotide is incapable of initiating an extension reaction (e.g., the 3′ end of the second bridging oligonucleotide is blocked), and/or the oligonucleotide probe is incapable of initiating an extension reaction (e.g., the 3′-end of the oligonucleotide probe is blocked).

In certain embodiments, the second region of the second bridging oligonucleotide is capable of annealing to a complementary sequence or partial sequence thereof of the consensus sequence B of the primer B.

In certain embodiments, the second region of the second bridging oligonucleotide is located at the 3′-end of the second bridging oligonucleotide.

In certain embodiments, the first region of the second bridging oligonucleotide is located at the 5′-end of the second bridging oligonucleotide.

In certain embodiments, the second bridging oligonucleotide comprises a 5′ phosphate at the 5′-end.

In certain embodiments, the second bridging oligonucleotide comprises a free —OH at the 3′-end.

In certain embodiments, the first bridging oligonucleotide is incapable of initiating an extension reaction (e.g., the 3′-end of the first bridging oligonucleotide is blocked).

In certain embodiments, the kit comprises: the nucleic acid array for labeling nucleic acids as described in (i), and the primer set comprising the primer A and primer B as described in (ii).

In certain embodiments, the capture sequence A of the primer A is a random oligonucleotide sequence.

In certain embodiments, the capture sequence A of the primer A is a poly(T) sequence or a sequence specific for a target nucleic acid. In certain embodiments, the primer A further comprises a consensus sequence A and optionally a tag sequence A, such as a random oligonucleotide sequence. In certain embodiments, the capture sequence A is located at the 3′-end of the primer A, and the consensus sequence A is located upstream (e.g., located at the 5′-end) of the primer A.

In certain embodiments, the primer B comprises a consensus sequence B, a complementary sequence of the 3′-end overhang, and a tag sequence B.

In certain embodiments, the primer B comprises a modified nucleotide (e.g., locked nucleic acid). In certain embodiments, the primer B comprises one or more modified nucleotides (e.g., one or more locked nucleic acids) at the 3′-end.

Optionally, the oligonucleotide probe is capable of initiating an extension reaction (e.g., the oligonucleotide probe comprises a free —OH at the 3′-end) or is incapable of initiating an extension reaction (e.g., the 3′-end of the oligonucleotide probe is blocked).

In certain embodiments, the kit comprises: the nucleic acid array for labeling nucleic acids as described in (i), the primer set comprising the primer A′ and primer B′ as described in (ii), and, (iii) a first bridging oligonucleotide and a second bridging oligonucleotide; wherein the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence A of the primer A′.

In certain embodiments, the capture sequence A of the primer A′ is a random oligonucleotide sequence.

In certain embodiments, the capture sequence A of the primer A′ is a poly(T) sequence or a sequence specific for a target nucleic acid. In certain embodiments, the primer A′ further comprises a tag sequence A, such as a random oligonucleotide sequence. In certain embodiments, the capture sequence A is located at the 3′-end of the primer A′, and the consensus sequence A is located upstream of the tag sequence A (e.g., located at the 5′-end of the primer A′).

In certain embodiments, the primer B′ comprises a modified nucleotide (e.g., a locked nucleic acid). In certain embodiments, the primer B′ comprises one or more modified nucleotides (e.g., one or more locked nucleic acids) at the 3′-end.

In certain embodiments, the kit further comprises a primer B″ or a random primer, in which the primer B″ is capable of annealing to a complementary sequence or partial sequence thereof of the consensus sequence B, and initiating an extension reaction.

In certain embodiments, the second region of the second bridging oligonucleotide is capable of annealing to a complementary sequence or partial sequence thereof (e.g., the 3′-end partial sequence thereof) of the consensus sequence A of the primer A′.

In certain embodiments, the second region of the first bridging oligonucleotide is located at the 3′-end of the first bridging oligonucleotide.

In certain embodiments, the first region of the first bridging oligonucleotide is located at the 5′-end of the first bridging oligonucleotide.

In certain embodiments, the first bridging oligonucleotide comprises a 5′ phosphate at the 5′-end.

In certain embodiments, the first bridging oligonucleotide comprises a free —OH at the 3′-end.

In certain embodiments, the second bridging oligonucleotide is incapable of initiating an extension reaction (e.g., the 3′-end of the second bridging oligonucleotide is blocked), and/or the oligonucleotide probe is incapable of initiating an extension reaction (e.g., the 3′-end of the oligonucleotide probe is blocked).

In certain embodiments, the second region of the second bridging oligonucleotide is capable of annealing to a complementary sequence or partial sequence thereof of the consensus sequence A of the primer A′.

In certain embodiments, the second region of the second bridging oligonucleotide is located at the 3′-end of the second bridging oligonucleotide.

In certain embodiments, the first region of the second bridging oligonucleotide is located at the 5′-end of the second bridging oligonucleotide.

In certain embodiments, the second bridging oligonucleotide comprises a 5′ phosphate at the 5′-end.

In certain embodiments, the second bridging oligonucleotide comprises a free —OH at the 3′-end.

In certain embodiments, the first bridging oligonucleotide is incapable of initiating an extension reaction (e.g., the 3′-end of the first bridging oligonucleotide is blocked).

In certain embodiments, the kit comprises: the nucleic acid array for labeling nucleic acids as described in (i), and the primer set comprising the primer A′ and primer B′ as described in (ii).

In certain embodiments, the capture sequence A of the primer A′ is a random oligonucleotide sequence.

In certain embodiments, the capture sequence A of the primer A′ is a poly(T) sequence or a sequence specific for a target nucleic acid. In certain embodiments, the primer A′ further comprises a tag sequence A, such as a random oligonucleotide sequence. In certain embodiments, the capture sequence A is located at the 3′-end of the primer A′, and the consensus sequence A is located upstream of the tag sequence A (e.g., located at the 5′-end of the primer A′).

In certain embodiments, the primer B′ comprises a consensus sequence B, a complementary sequence of the 3′-end overhang, and a tag sequence B.

In certain embodiments, the primer B′ comprises a modified nucleotide (e.g., locked nucleic acid). In certain embodiments, the primer B′ comprises one or more modified nucleotides (e.g., one or more locked nucleic acids) at the 3′-end.

In certain embodiments, the kit further comprises a primer B″ or a random primer, in which the primer B″ is capable of annealing to a complementary sequence or partial sequence thereof of the consensus sequence B, and initiating an extension reaction.

Optionally, the oligonucleotide probe is capable of initiating an extension reaction (e.g., the oligonucleotide probe comprises a free —OH at the 3′-end) or incapable of initiating an extension reaction (e.g., the 3′-end of the oligonucleotide probe is blocked).

In certain embodiments, the kit has one or more characteristics selected from the following:
(1) the oligonucleotide probe, primer A, primer A′, primer B, primer B′, primer B″, random primer, first bridging oligonucleotide, and second bridging oligonucleotide each independently comprise or consist of natural nucleotides (e.g., deoxyribonucleotides or ribonucleotides), modified nucleotides, non-natural nucleotides, or any combination thereof;
(2) the oligonucleotide probes each independently have a length of 15 to 300 nt (e.g., 15 to 200 nt, 15 to 20 nt, 20 to 30 nt, 30 to 40 nt, 40 to 50 nt, 50 to 100 nt, 100 to 150 nt, 150 to 200 nt);
(3) the primer A, primer A′, primer B, primer B′, primer B″, and random primer each independently have a length of 4 to 200 nt (e.g., 5 to 200 nt, 15 to 230 nt, 26 to 115 nt, 10 to 130 nt, 10 to 20 nt, 20 to 50 nt, 20 to 30 nt, 30 to 40 nt, 40 to 50 nt, 50 to 100 nt, 100 to 150 nt, 150 to 200 nt);
(4) the first bridging oligonucleotide and the second bridging oligonucleotide each independently have a length of 6 to 200 nt (e.g., 20 to 100 nt, 20 to 70 nt, 6 to 15 nt, 15 to 20 nt, 20 to 30 nt, 30 to 40 nt, 40 to 50 nt, 50 to 100 nt, 100 to 150 nt, 150 to 200 nt); (5) the oligonucleotide probes coupled to the same solid support have the same consensus sequence X1 and/or the same consensus sequence X2;
(6) the consensus sequence X1 of the oligonucleotide probes comprises a cleavage site; in some embodiments, the cleavage site can be cleaved or broken by a method selected from nicking enzyme digestion, USER enzyme digestion, light-responsive excision, chemical excision or CRISPR-mediated excision.

In certain embodiments, the kit further comprises a reverse transcriptase, a nucleic acid ligase, a nucleic acid polymerase and/or a transposase.

In certain embodiments, the reverse transcriptase has terminal deoxynucleotidyl transferase activity. In certain embodiments, the reverse transcriptase can synthesize a cDNA strand using an RNA (e.g., mRNA) as a template, and add an overhang to the 3′-end of the cDNA strand. In certain embodiments, the reverse transcriptase is capable of adding to the 3′-end of the cDNA strand an overhang having a length of at least 1, at least 2, at least 3, at least 4, at least 5, at least 6, at least 7, at least 8, at least 9, at least 10 or more nucleotides. In certain embodiments, the reverse transcriptase is capable of adding to the 3′-end of the cDNA strand an overhang of 2-5 cytosine nucleotides (e.g., a CCC overhang). In certain embodiments, the reverse transcriptase is selected from the group consisting of M-MLV reverse transcriptase, HIV-1 reverse transcriptase, AMV reverse transcriptase, telomerase reverse transcriptase, and variants, modified products and derivatives thereof having the reverse transcriptase activity of the above-mentioned reverse transcriptases.

In certain embodiments, the nucleic acid polymerase does not have 5′ to 3′ exonucleolytic activity or strand displacement activity.

In certain embodiments, the nucleic acid polymerase has 5′ to 3′ exonucleolytic activity or strand displacement activity.

In certain embodiments, the transposase is selected from the group consisting of Tn5 transposase, MuA transposase, Sleeping Beauty transposase, Mariner transposase, Tn7 transposase, Tn10 transposase, Ty1 transposase, Tn552 transposase, as well as variants, modified products and derivatives thereof having the transposase activity of the above-mentioned transposases.

In certain embodiments, the kit further comprises: the primer C, the primer D, the primer C′, and/or the primer D′. For example, the kit further comprises the primer C, the primer D and the primer D′. For example, the kit further comprises the primer C, the primer D, the primer C′ and the primer D′.

In certain embodiments, the kit further comprises: a reagent for nucleic acid hybridization, a reagent for nucleic acid extension, a reagent for nucleic acid amplification, a reagent for recovering or purifying nucleic acid, a reagent for constructing a transcriptome sequencing library, a reagent for sequencing (e.g., second- or third-generation sequencing), or any combination thereof.

### Use

In another aspect, the present application also provides a use of the above method for generating a population of labeled nucleic acid molecules or the above kit for constructing a library of nucleic acid molecules or for performing transcriptome sequencing.

### DEFINITION OF TERMS

In the present application, unless otherwise stated, scientific and technical terms used herein have the meanings commonly understood by those skilled in the art. Moreover, the operating steps of molecular biology, biochemistry, nucleic acid chemistry, cell culture, etc., as used herein are all routine steps widely used in the corresponding fields. Meanwhile, in order to better understand the present application, definitions and explanations of relevant terms are provided below.

When the terms “e.g.,” “for example,” “such as,” “comprise,” “include,” or variants thereof are used herein, these terms will not be considered as limiting terms and will instead be interpreted to mean “but not limited” or “without limitation.”

Unless otherwise indicated herein or clearly contradicted by context, the terms “a” and “an” as well as “the” and similar referents in the context of describing the present application (especially in the context of the following claims) are to be construed to cover singular and plural.

As used in this application, “DNB” (DNA nanoball) is a typical RCA (rolling circle amplification) product, which has the characteristics of RCA products. Wherein, the RCA product is a single-stranded DNA sequence with multiple copies, which can form a similar “spherical” structure due to the interaction between the bases comprised in the DNA. Typically, a library molecule is circularized to form a single-stranded circular DNA, and subsequently the single-stranded circular DNA can be amplified by multiple orders of magnitude using the rolling circle amplification technology, thereby generating an amplification product called DNB.

As used herein, a “nucleic acid molecule population” refers to a population or collection of nucleic acid molecules, for example, nucleic acid molecules derived directly or indirectly from a target nucleic acid molecule (e.g., a double-stranded DNA, an RNA/cDNA hybrid, a single-stranded DNA, or a single-stranded RNA). In some embodiments, a nucleic acid molecule population comprises a library of nucleic acid molecules, and the library of nucleic acid molecules comprises sequences that are qualitatively and/or quantitatively representative of a target nucleic acid molecule sequence. In other embodiments, the population of nucleic acid molecules comprises a subset of the library of nucleic acid molecules.

As used herein, a “library of nucleic acid molecules” refers to a collection or population of labeled nucleic acid molecules (e.g., labeled double-stranded DNA, labeled RNA/cDNA hybrid, labeled single-stranded DNA, or labeled single-stranded RNA) or fragments thereof that are generated directly or indirectly from a target nucleic acid molecule, wherein the combination of labeled nucleic acid molecules or fragments thereof in the collection or population is shown to be qualitatively and/or quantitatively representative of the sequence of a target nucleic acid molecule sequence from which the labeled nucleic acid molecules are generated. In certain embodiments, the library of nucleic acid molecules is a sequencing library. In certain embodiments, the library of nucleic acid molecules can be used to construct a sequencing library.

As used herein, a “cDNA” or “cDNA strand” refers to a “complementary DNA” synthesized by extension using at least a portion of an RNA molecule of interest as a template via a primer that anneals to the RNA molecule of interest under catalysis of an RNA-dependent DNA polymerase or reverse transcriptase (this process is also called “reverse transcription”). The synthesized cDNA molecule is “homologous” to or “complementary” to or “base pairs” with or “forms a complex” with at least a portion of the template.

As used herein, the term “upstream” is used to describe the relative positional relationship of two nucleic acid sequences (or two nucleic acid molecules) and has a meaning commonly understood by those skilled in the art. For example, the expression “a nucleic acid sequence is located upstream of another nucleic acid sequence” means that, when aligned in the 5′ to 3′ direction, the former is located at a more forward position (i.e., a position closer to the 5′-end) than the latter. As used herein, the term “downstream” has the opposite meaning to “upstream.” As used herein, a “tag sequence Y”, “tag sequence A”, “tag sequence B”, “consensus sequence X1”, “consensus sequence X2”, “consensus sequence A”, “consensus sequence B”, etc., refer to an oligonucleotide having non-target nucleic acid components that provide a means of identification, recognition, and/or molecular manipulation or biochemical manipulation (e.g., by providing a site for annealing an oligonucleotide, the oligonucleotide being, for example, a primer for DNA polymerase extension or an oligonucleotide for capture reaction or ligation reaction) for a nucleic acid molecule ligated thereto or a derivative product of the nucleic acid molecule ligated thereto (e.g., a complementary fragment of the nucleic acid molecule, a short fragment of the nucleic acid molecule, etc.). The oligonucleotide may consist of at least two (preferably about 6 to 100, but there is no definite limit to the length of the oligonucleotide, and the exact size depends on many factors, while these factors in turn depend on the final function or use of the oligonucleotide) continuous nucleotides, and can also be composed of multiple oligonucleotide fragments in a continuous or non-continuous arrangement. The oligonucleotide sequence may be unique to each nucleic acid molecule to which it is ligated, or may be unique to a certain type of nucleic acid molecule to which it is ligated. The oligonucleotide sequence may be reversibly or irreversibly ligated to a polynucleotide sequence to be “labeled” by any method including ligation, hybridization or other methods. The process of ligating the oligonucleotide sequence to a nucleic acid molecule is sometimes referred to herein as “labeling”, and a nucleic acid molecule that undergoes the addition of a label or comprises a label sequence is called a “labeled nucleic acid molecule” or “tagged nucleic acid molecule.”

For various reasons, the nucleic acid or polynucleotide of the present application (e.g., “tag sequence Y”, “tag sequence A”, “tag sequence B”, “consensus sequence X1”, “consensus sequence X2”, “consensus sequence A”, “consensus sequence B”, “primer A”, “primer A′”, “primer B”, “primer B′”, “primer B″”, “primer C”, “primer D”, “primer D′”, “random primer”, “first bridging oligonucleotide”, “second bridging oligonucleotide”, etc.) may comprise one or more modified nucleic acid bases, sugar moieties, or internucleoside linkages. For example, some reasons for using nucleic acids or polynucleotides comprising modified bases, sugar moieties, or internucleoside linkages include, but are not limited to: (1) changes in Tm; (2) changes in the susceptibility of a polynucleotide to one or more nucleases; (3) providing a moiety for linking a label; (4) providing a label or label quencher; or (5) providing a moiety such as biotin for attaching another molecule in solution or bound to a surface. For example, in some embodiments, oligonucleotides such as primers can be synthesized such that the random portions comprise one or more nucleic acid analogs with constrained conformation, including, but not limited to, one or more ribonucleic acid analogs in which ribose ring is “locked” by the methylene bridge that links the 2′-0 atom to the 4′-C atom; these modified nucleotides result in an increase in the Tm, or melting temperature, of each molecule by about 2 degrees Celsius to about 8 degrees Celsius. For example, in some embodiments in which an oligonucleotide primer comprising ribonucleotides is used, one indicator of using a modified nucleotide in the method may be that the oligonucleotide comprising the modified nucleotide may be digested by a single-strand specific RNase.

In the methods of the present application, for example, the nucleic acid bases in a single nucleotide at one or more positions in a polynucleotide or oligonucleotide may comprise guanine, adenine, uracil, thymine or cytosine; or optionally, one or more of the nucleic acid bases may comprise modified bases such as, but not limited to, xanthine, allylamino-uracil, allylamino-thymine nucleoside, hypoxanthine, 2-aminoadenine, 5-propynyluracil, 5-propynylcytosine, 4-thiouracil, 6-thioguanine, azauracil and deazauracil, thymine nucleoside, cytosine, adenine or guanine. Furthermore, they may comprise nucleic acid bases derivatized with the following moieties: biotin moiety, digoxigenin moiety, fluorescent or chemiluminescent moiety, quenching moiety or some other moieties. The present application is not limited to the listed nucleic acid bases; the list given illustrates examples of a wide range of bases that may be used in the methods of the present application.

With respect to the nucleic acids or polynucleotides of the present application, one or more of the sugar moieties may comprise 2′-deoxyribose, or optionally, one or more of the sugar moieties may comprise some other sugar moieties, such as, but not limited to: ribose or 2′-fluoro-2′-deoxyribose or 2′-O-methyl-ribose that possesses resistance to some nucleases, or 2′-amino-2′-deoxyribose or 2′-azido-2′-deoxyribose that is labeled by reaction with a visible, fluorescent, infrared fluorescent or other detectable dye or a chemical substance with an electrophilic, photoreactive, alkynyl or other reactive chemical moiety.

The internucleoside linkages of the nucleic acids or polynucleotides of the present application may be phosphodiester linkages, or optionally, one or more of the internucleoside linkages may comprise modified linkages such as, but not limited to: phosphorothioate, phosphorodithioate, phosphoroselenate, or phosphorodiselenate linkages, which are resistant to some nucleases.

As used herein, the term “terminal deoxynucleotidyl transferase activity” refers to an ability to catalyze the template-independent addition (or “tailing”) of one or more deoxyribonucleoside triphosphates (dNTPs) or single dideoxyribonucleoside triphosphates to the 3′-end of cDNA. Examples of reverse transcriptases with terminal deoxynucleotidyl transferase activity comprise, but are not limited to, M-MLV reverse transcriptase, HIV-1 reverse transcriptase, AMV reverse transcriptase, telomerase reverse transcriptase, and variants, modified products and derivatives thereof with the reverse transcription activity and terminal deoxynucleotidyl transferase activity of the reverse transcriptases. The reverse transcriptases have or do not have RNase activity (especially RNase H activity). In preferred embodiments, the reverse transcriptases used for the reverse transcription of RNA to generate cDNA do not have RNase activity (especially RNase H activity). Therefore, in a preferred embodiment, the reverse transcriptase used for the reverse transcription of RNA to generate cDNA has terminal deoxynucleotidyl transferase activity, and does not have RNase activity (especially RNase H activity).

As used herein, a nucleic acid polymerase with “strand displacement activity” refers to a nucleic acid polymerase that, during the process of extending a new nucleic acid strand, if it encounters a downstream nucleic acid strand complementary to the template strand, can continue the extension reaction and replace (rather than degrade) the nucleic acid strand that is complementary to the template strand.

As used herein, a nucleic acid polymerase having “5′ to 3′ exonucleolytic activity” refers to a nucleic acid polymerase that can catalyze the hydrolysis of 3,5-phosphodiester bonds in the order of 5′ to 3′ of polynucleotide, thereby degrading nucleotides.

As used herein, a nucleic acid polymerase (or DNA polymerase) with “high fidelity” refers to a nucleic acid polymerase (or DNA polymerase) that has a lower probability of introducing erroneous nucleotides (i.e., an error rate) during the amplification of nucleic acids than the wild-type Taq enzyme (e.g., the Taq enzyme whose sequence is shown in UniProt Accession: P19821.1).

As used herein, the terms “annealed,” “annealing,” “anneal,” “hybridized,” or “hybridizing” and the like refer to the formation of complex between nucleotide sequences having sufficient complementarity to form complex via Watson-Crick base pairing. For the purposes of the present application, nucleic acid sequences that “are complementary” or “hybridize” or “anneal” to each other should be capable of forming a sufficiently stable “hybrid” or “complex” for the intended purpose. It is not required that every nucleic acid base within the sequence displayed by a nucleic acid molecule is capable of base pairing or pairing or complexing with every nucleic acid base within the sequence displayed by another nucleic acid molecule such that both nucleic acid molecules or corresponding sequences displayed therein “are complementary” or “anneal” or “hybridize” to each other. As used herein, the term “complementary” or “complementarity” is used when referring to nucleotide sequences that are related by the rules of base pairing. For example, the sequence 5′-A-G-T-3′ is complementary to the sequence 3′-T-C-A-5′. Complementarity can be “partial”, in which only some of the nucleic acid bases match according to the rules of base pairing. Optionally, there may be “complete” or “total” complementarity between nucleic acids. The degree of complementarity between nucleic acid strands has a significant impact on the efficiency and strength of hybridization between nucleic acid strands. The degree of complementarity is particularly important in amplification reactions and detection methods that rely on hybridization of nucleic acids. The term “homology” refers to the degree of complementarity of one nucleic acid sequence to another nucleic acid sequence. There may be partial homology (i.e., complementarity) or complete homology (i.e., complementarity). A partially complementary sequence is a sequence that at least partially inhibits hybridization of a fully complementary sequence to a target nucleic acid and is referred to using the functional term “substantially homologous”. Inhibition of hybridization of a fully complementary sequence to a target sequence can be tested under low stringency conditions using hybridization assays (e.g., Southern blotting or Northern blotting, hybridization in solution, etc.). Substantially homologous sequences or probes will compete in or inhibit binding (i.e., hybridization) of fully homologous sequences to the target under low stringency conditions. This is not to say that low stringency conditions are conditions that allow for nonspecific binding; low stringency conditions require that the two sequences bind to each other via a specific (i.e., selective) interaction. The absence of non-specific binding can be tested by using a second target that lacks complementarity or has only a low degree of complementarity (e.g., less than about 30% complementarity). In cases where specific binding is low or absent, a probe will not hybridize to the nucleic acid target. When used in reference to a double-stranded nucleic acid sequence such as a cDNA or genomic clone, the term “substantially homologous” means it is any oligonucleotide or probe that can hybridize to one or both strands of the double-stranded nucleic acid sequence under the low stringency conditions described herein. As used herein, the terms “annealing” or “hybridization” are used when referring to the pairing of complementary nucleic acid strands. Hybridization and hybridization strength (i.e., strength of association between nucleic acid strands) are affected by many factors known in the art, including the degree of complementarity between nucleic acids, including the stringency of conditions affected by factors such as salt concentration, the Tm (melting temperature) to form a hybrid, the presence of other components (e.g., the presence or absence of polyethylene glycol or betaine), the molar concentration of hybridized strands, and the G:C content of nucleic acid strands.

As described herein, the solid support is capable of releasing the oligonucleotide probe spontaneously or upon exposure to one or more stimuli (e.g., temperature changes, pH changes, exposure to specific chemicals or phases, exposure to light, exposure to reducing agents, etc.). It will be appreciated that the oligonucleotide probe can be released by cleavage of the bond between the oligonucleotide probe and the solid support, or degradation of the solid support itself, or both, and the oligonucleotide probe allows being approached or can be approached by other reagents.

Adding multiple types of labile bonds to the solid support enables the capability of the solid support to respond to different stimuli. Each type of labile bond can be sensitive to an associated stimulus (e.g., chemical stimulus, light, temperature, etc.) such that the release of a substance attached to the solid support through each labile bond can be controlled by applying an appropriate stimulus. In addition to thermally cleavable bonds, disulfide bonds, and UV-sensitive bonds, other non-limiting examples of labile bonds that can be coupled to the solid support comprise ester bonds (e.g., ester bonds that can be cleaved with acids, bases, or hydroxylamine), ortho diol bonds (e.g., ortho diol bonds that can be cleaved by sodium periodate), Diels-Alder bonds (e.g., Diels-Alder bonds that can be cleaved thermally), sulfone bonds (e.g., sulfone bonds that can be cleaved by alkali), silicyl ether bonds (e.g., silicyl ether bonds that can be cleaved by acids), glycosidic bonds (e.g., glycosidic bonds that can be cleaved by amylases), peptide bonds (e.g., peptide bonds that can be cleaved by proteases), or phosphodiester bonds (e.g., phosphodiester bonds that can be cleaved by nucleases (e.g., DNA enzyme)).

In addition to or as an alternative to the cleavable bonds between the solid support and the oligonucleotide described above, the solid support can be degradable, destructible or soluble spontaneously or upon exposure to one or more stimuli (e.g., temperature changes, pH changes, exposure to specific chemical substances or phases, exposure to light, exposure to reducing agents, etc.). In some cases, the solid support may be soluble such that the material components of the solid support dissolve upon exposure to specific chemicals or environmental changes (e.g., changes in temperature or changes in pH). In some cases, the solid support may degrade or dissolve under elevated temperatures and/or alkaline conditions. In some cases, the solid support may be thermally degradable such that the solid support degrades when exposed to appropriate temperature changes (e.g., heating). Degradation or dissolution of the solid support bound with a substance (e.g., an oligonucleotide probe) can result in the release of the substance from the solid support.

As used herein, the terms “transposase” and “reverse transcriptase” and “nucleic acid polymerase” refer to a protein molecule or an aggregate of protein molecules responsible for catalyzing specific chemical and biological reactions. In general, the methods, compositions or kits of the present application are not limited to the use of a specific transposase, reverse transcriptase or nucleic acid polymerase from a specific source. Rather, the methods, compositions, or kits of the present application may comprise any transposases, reverse transcriptases, or nucleic acid polymerases from any sources that have equivalent enzymatic activity to the specific enzymes of the specific methods, compositions, or kits disclosed herein. Furthermore, the methods of the present application also comprise the following embodiments: wherein any one specific enzyme provided and used in the steps of the methods is replaced by a combination of two or more enzymes, when the two or more enzymes are used in combination, whether used separately in a stepwise manner or together simultaneously, the reaction mixtures produce the same results as would be obtained using that specific enzyme. The methods, buffers, and reaction conditions provided herein, including those in the Examples, are currently preferred for embodiments of the methods, compositions, and kits of the present application. However, that other enzyme storage buffers, reaction buffers, and reaction conditions can be used for some of the enzymes of the present application are known in the art and may also be suitable for use in the present application and are comprised herein.

### Beneficial Effects of the Present Application

The present application provides a new method for generating a population of labeled nucleic acid molecules, as well as a method for constructing a library of nucleic acid molecules based on the method and performing a high-throughput sequencing, thereby achieving high-precision subcellular-level spatial positioning of samples. The method of the present application has one or more beneficial technical effects selected from the following:

(1) The probes of traditional nucleic acid arrays (e.g., chips) used for spatial transcriptome sequencing comprise fixed capture sequences. Usually a specific capture sequence can only capture the specific target nucleic acid molecule corresponding to it. For example, when the capture sequence is a poly (T), it correspondingly captures a target nucleic acid molecule comprising poly(A). If the target nucleic acid molecule changes, the probe sequence comprising the capture sequence needs to be changed accordingly, that is, the entire nucleic acid array (e.g., a chip) needs to be changed, which is costly and inefficient in practical applications. The nucleic acid array (e.g., a chip) of the present application does not comprise a capture sequence, and the capture sequence exists in a reverse transcription primer that is independent of the nucleic acid array (that is, the capture sequence and the probe are independent of each other). After the capture sequence captures the target nucleic acid molecule, it ligates to the probe via a bridging oligonucleotide.

Therefore, the present application can design corresponding capture sequences for different target nucleic acid molecules without changing the probe sequence (that is, without changing the nucleic acid array (e.g., a chip)), and achieve the capture of different target nucleic acid molecules by changing the capture sequences and the bridging oligonucleotides.

(2) Traditional spatial transcriptome methods all use poly(T) as a capture sequence and cannot capture RNA without poly(A) tail, while the present application can achieve the capture of target nucleic acid molecules without poly(A) tail by replacing the poly(T) in the capture sequence with a continuous random sequence group (e.g., a random primer sequence, such as N6, N8, etc.), and, the continuous random sequence group can also serve as a unique molecular identifier (UMI) sequence at the same time.

(3) Traditional nucleic acid arrays (e.g., chips) used for spatial transcriptome sequencing have fixed capture probes. Generally, tissue permeabilization is performed first to release intracellular RNA. If permeabilization is excessive, RNA will spread to adjacent cells, even the periphery of the tissue sample, and is captured by the probes, making it impossible to achieve in-situ capture of mRNA. If permeabilization is incomplete, the capture efficiency of mRNA will be affected. With the method of the present application, the nucleic acid array (e.g., a chip) does not comprise a capture sequence (the nucleic acid array comprises spatial information and does not comprise a capture sequence), and the purpose of tissue permeabilization is to allow the reverse transcription primer to enter the cell and hybridize with the mRNA in situ, without the need for intense permeabilization reagent treatment, thereby reducing sample spread.

The preferred embodiments of the present application are described in detail below in conjunction with the accompanying drawings and examples, but those skilled in the art will understand that the following drawings and examples are only used to illustrate the present application and do not limit the scope of the present application. The various objects and advantageous aspects of the present application will become apparent to those skilled in the art from the accompanying drawings and the following detailed description of preferred embodiments.

### Specific Models for Carrying Out the Application

The present application will now be described with reference to the following examples which are intended to illustrate (but not to limit) the present application. Unless otherwise indicated, the experiments and methods described in the examples were performed essentially according to conventional methods well known in the art and described in various references. In addition, if the specific conditions are not specified in the examples, the conventional conditions or the conditions recommended by the manufacturer should be followed. If the manufacturer of the reagents or instruments used was not indicated, they were all conventional products that could be purchased commercially. Those skilled in the art will appreciate that the examples describe the present application by way of example and are not intended to limit the scope sought to be protected by the present application. All publications and other references mentioned herein are incorporated by reference in their entirety.

### BRIEF DESCRIPTION OF THE DRAWINGS

FIG. 1 shows an exemplary structure of a chip used for capturing and labeling nucleic acid molecules in the present application, which comprises: a chip and oligonucleotide probes (also called chip sequences) coupled to the chip. Each kind of oligonucleotide probe comprises a tag sequence Y corresponding to its position on the chip, and a region with one kind of oligonucleotide probe coupled to the chip can be called a microdot. Each kind of oligonucleotide probe may comprise a single copy or multiple copies.

FIG. 2 shows an exemplary scheme for preparing a cDNA strand using an RNA (e.g., mRNA) in a sample as a template, as well as an exemplary structure of the cDNA strand. CA: consensus sequence A; CB: consensus sequence B.

FIG. 3 shows an exemplary scheme 1 for labeling the 3-end of a cDNA strand with a complementary sequence of the chip sequence to form a new nucleic acid molecule comprising the chip sequence information (i.e., a nucleic acid molecule labeled by the chip sequence), and, an exemplary structure of the new nucleic acid molecule comprising the chip sequence information. CA: consensus sequence A; CB: consensus sequence B; X1: consensus sequence X1; Y: tag sequence Y; X2: consensus sequence X2; P1: first region; P2: second region.

FIG. 4 shows an exemplary scheme 2 for labeling the 3-end of a cDNA strand with a complementary sequence of the chip sequence to form a new nucleic acid molecule comprising the chip sequence information (i.e., a nucleic acid molecule labeled by the chip sequence), and, an exemplary structure of the new nucleic acid molecule comprising the chip sequence information. CA: consensus sequence A; CB: consensus sequence B; X1: consensus sequence X1; Y: tag sequence Y; X2: consensus sequence X2.

FIG. 5 shows an exemplary scheme for preparing a complementary strand of a cDNA strand using an RNA (e.g., mRNA) in a sample as a template, and an exemplary structure of the complementary strand of the cDNA strand. CA: consensus sequence A; CB: consensus sequence B; EP: extension primer.

FIG. 6 shows an exemplary scheme 1 for labeling the 3-end of a complementary strand of a cDNA strand with a complementary sequence of a chip sequence to form a new nucleic acid molecule comprising the chip sequence information (i.e., a nucleic acid molecule labeled by the chip sequence), and, an exemplary structure of the new nucleic acid molecule comprising the chip sequence information. CA: consensus sequence A; CB: consensus sequence B; X1: consensus sequence X1; Y: tag sequence Y; X2: consensus sequence X2; P1: first region; P2: second region.

FIG. 7 shows an exemplary scheme 2 for labeling the 3-end of a complementary strand of a cDNA strand with a complementary sequence of a chip sequence to form a new nucleic acid molecule comprising the chip sequence information (i.e., a nucleic acid molecule labeled by the chip sequence), and, an exemplary structure of the new nucleic acid molecule comprising the chip sequence information. CA: consensus sequence A; CB: consensus sequence B; X1: consensus sequence X; Y: tag sequence Y; X2: consensus sequence X2.

FIG. 8 shows the length distribution of the cDNA amplification product prepared in Example 2.

FIG. 9 shows the spatial gene expression map of the mouse brain section obtained by sequencing analysis in Example 3.

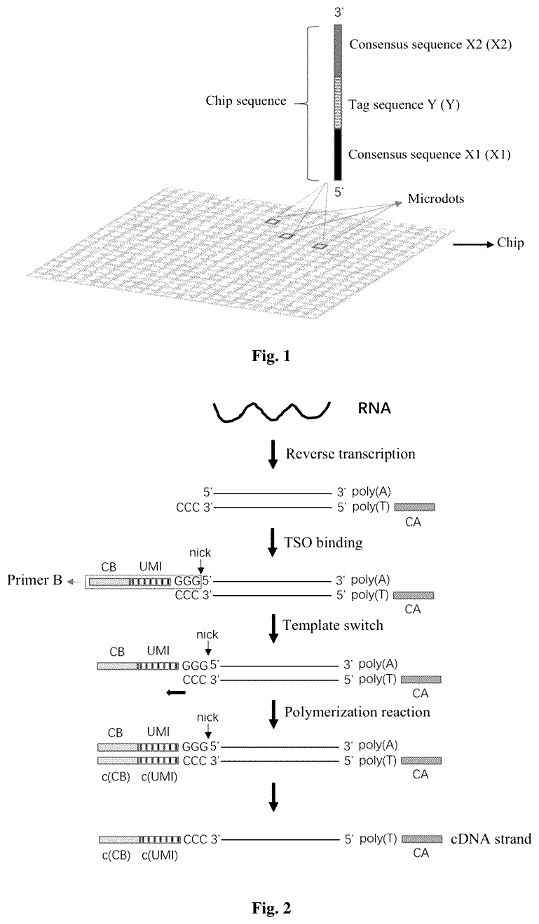

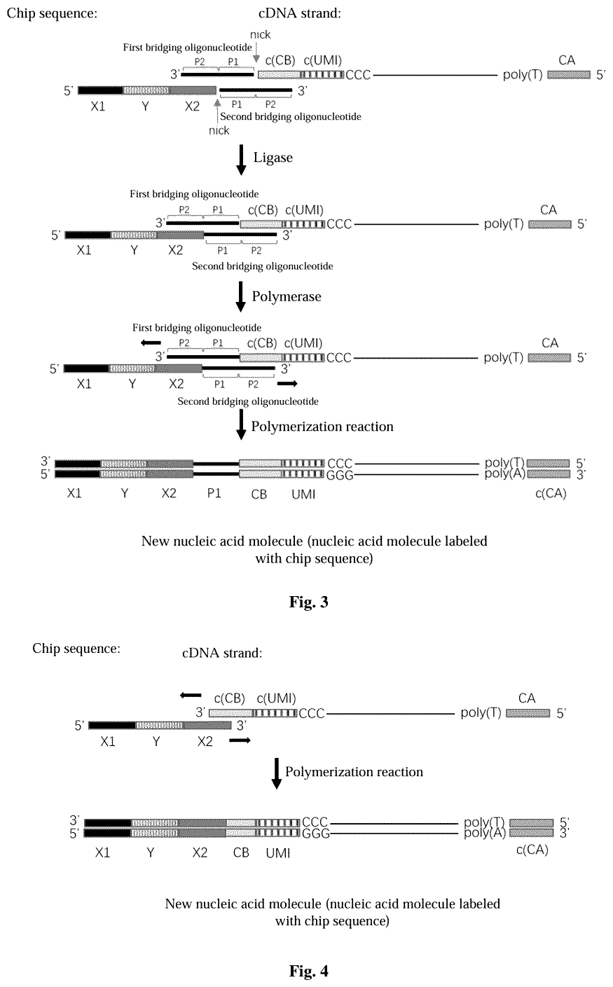

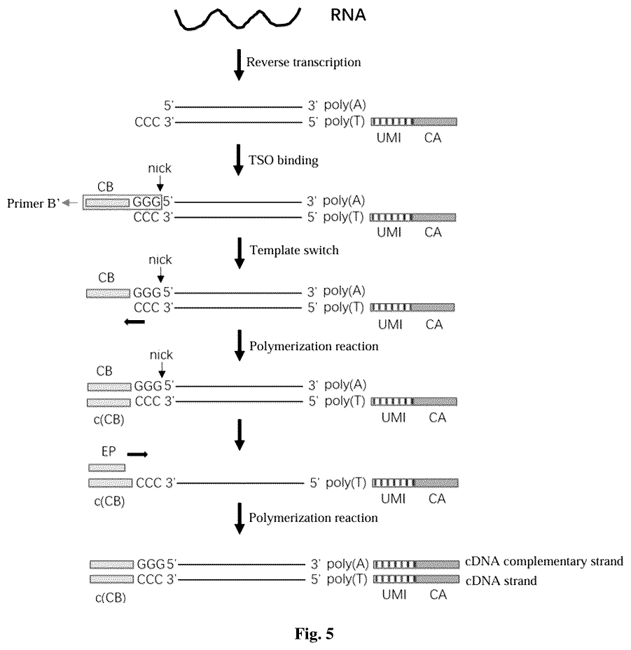

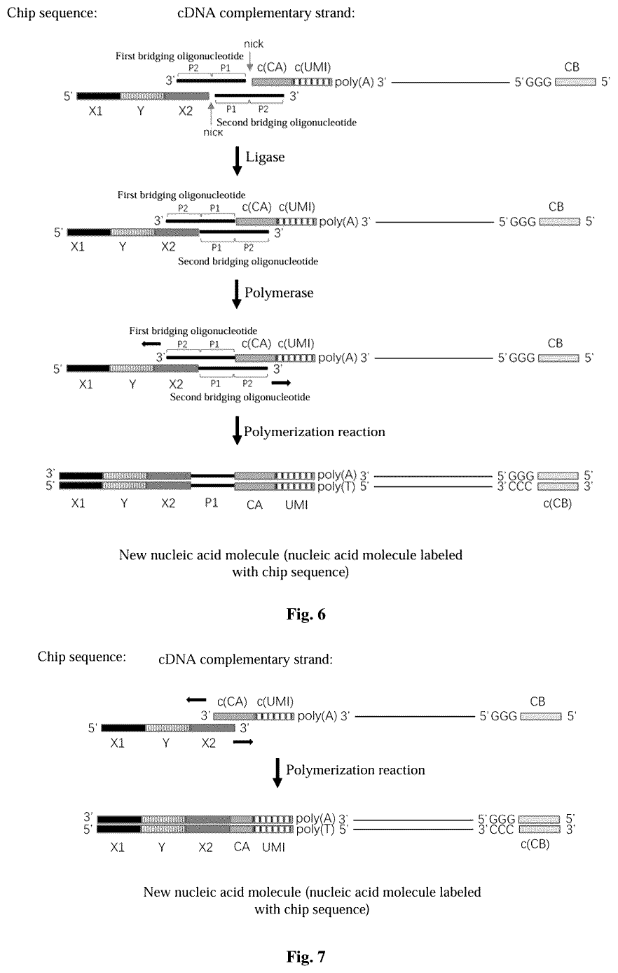

### SEQUENCE INFORMATION

Information on some of the sequences involved in the present application is provided in Table 1 below.

TABLE 1
Sequence information

| SEQ | | |
|---|---|---|
| ID NO | Description | Sequence information |
| 1 | Nucleotide sequence of DNA | p-GAACGACATGGCTACGATCCGACTTN |
| | library molecule | NNNNNNNNNNNNNNNNAAGTCGGAGGCC |
| | | AAGCGGTCTTAGGAAGACAANNNNNNNN |
| | | NNNNNNNNNNNNNNNNNCTGATAAGGTC |
| | | GCCATGCCTCTCAGTACGTCAGCAGTTN |
| | | NNNNNNNNNNNNNNNNCAACTCCTTGGC |
| | | TCACA |
| 2 | Nucleotide sequence of DNB | GCCATGTCGTTCGGAACCGAGTGT |
| | primer | |
| 3 | Nucleotide sequence of chip | CTGCTGACGTACTGAGAGGCATGGCGAC |
| | sequence synthesis primer | CTTATCAG |
| 4 | Consensus sequence X1 | CTGCTGACGTACTGAGAGGCATGGCGAC |
| | | CTTATCAG |
| 5 | Consensus sequence X2 | TTGTCTTCCTAAG |
| 6 | Nucleotide sequence of polyT | p-ACCGCTTGGCCTCCGTTTTTTTTTTT |
| | primer | TTTTTTTTTTTTTTTTTV |
| 7 | TSO sequence | AAGCAGTGGTATCAACGCAGAGTACATN |
| | | NNNNNrGrG+G |
| 8 | Chip sequence | CTGCTGACGTACTGAGAGGCATGGCGAC |
| | | CTTATCAGNNNNNNNNNNNNNNNNNNNN |
| | | NNNNNTTGTCTTCCTAAG |
| 9 | First bridging oligonucleotide | p-GGGGGGGGCTTAGGAAGACAA |
| | sequence | |
| 10 | Second bridging oligonucleotide | p-CCCCCCCCAAGCAGTGGTATCAA |
| | sequence | |
| 11 | Sequence of cDNA amplification | CTGCTGACGTACTGAGAGGCATG |
| | primer 1 | |
| 12 | Sequence of cDNA amplification | ACCGCTTGGCCTCCGTGCTATC |
| | primer 2 | |
| 13 | Library construction primer 1 | p-CTGCTGACGTACTGAGAGGC*A*T |
| 14 | Library construction primer 2 | GAGACGTTCTCGACTCAGAAGATG |
| 15 | Primer sequence to prepare DNB | GGCCTCCGACTTGAGACGTTCTCG |
| | for sequencing | |

Note:
“r” indicates that the nucleotide at the 3′ adjacent position is ribonucleotide;
“+” indicates that the nucleotide at the 3′ adjacent position comprises LNA (locked nucleotide);
“*” indicates phosphorothioate modification;
“p” indicates phosphorylation modification;
N = A, T, C or G;
V = A, C or G.

### Example 1: Preparation of Capture Chip

1. Sequences of DNA library molecules comprising the position information of a chip were designed, which comprised from 5′ to 3′: coding sequences of consensus sequence X1 (X1), tag sequence Y (Y) and consensus sequence X2 (X2). A typical nucleotide sequence of a DNA library molecule was shown in SEQ ID NO: 1. Beijing Liuhe BGI Co., Ltd. was entrusted to synthesize the DNA library molecules.

### 2. Amplification and Loading of Library Molecules

(1) DNBSEQ sequencing kit (purchased from MGI, Cat. No. 1000019840) was used to prepare DNA nano ball (DNB). Specific embodiment was briefly described below.

Briefly, 40 μL of the reaction system as shown in Table 2 was prepared. The reaction system was placed in a PCR machine and the reaction was performed according to the following reaction conditions: 95° C. for 3 minutes, 40° C. for 3 minutes. After the reaction was completed, the reaction product was placed on ice, added with 40 μL of mixed enzyme I and 2 μL of mixed enzyme II (from DNBSEQ sequencing kit), 1 μL of ATP (100 mM stock solution, obtained from Thermo Fisher), and 0.1 μL of T4 ligase (obtained from NEB, Cat. No.: M0202S). After mixing well, the above reaction system was placed in a PCR machine and reacted at 30° C. for 20 minutes to generate DNBs.

TABLE 2
Reaction system for preparing DNBs

| Ingredient | Volume (μL) | Final concentration |
|---|---|---|
| DNA library molecule | X (80 fmol) | — |
| 10X phi29 buffer | 4 | 1X |
| 10 μM DNB primer (SEQ ID NO: 2; synthesized | 4 | 1 μM |
| by Liuhe BGI) | | |
| H₂O | 32-x | — |

(2) Subsequently, the DNBs were loaded onto a BGISEQ 500 sequencing chip according to the method described in the BGISEQ 500 high-throughput sequencing reagent set (SE50) (purchased from MGI, Cat. No.: 1000012551).

In the sequencing chip, the MDA reagent in the BGISEQ 500 PE50 sequencing kit (purchased from MGI, 1000012554) was added, and incubated at 37° C. for 30 minutes, and then the chip was washed with 5×SSC.

(3) The chip surface was modified with N3-PEG3500-NHS (a modification reagent purchased from sigma, Cat. No.: JKA5086). After incubation for 30 minutes, the DBCO-modified primer for chip sequence synthesis (its sequence was shown in SEQ ID NO: 3) was pumped therein, and incubated overnight at room temperature. 3. Sequencing and decoding of position sequence information. The DNBs were sequenced according to the instructions of the BGISEQ-500 high-throughput sequencing reagent set, and the length of SE read was set as 25 bp. The above-mentioned DBCO-modified primer was extended to obtain a chain comprising position sequence information during the sequencing process, and the chain was decoded to obtain the position sequence information of corresponding DNB.

4. Continuously extension of the chain obtained in step 3 during the sequencing process: Based on the above step 3, the 15-base cPAS reaction was continued to obtain the chip sequence (SEQ ID NO: 8, which comprised a consensus sequence X1 (SEQ ID NO: 4), tag sequence Y and consensus sequence X2 (SEQ ID NO: 5)).

5. Restriction endonuclease HaeIII was used to excise the DNB, and the residual fragments on the DNB were removed by denaturation at high temperature so that only the chip sequences in step 4 remained on the chip.

6. Chip cutting: The prepared chip was cut into several small pieces. The size of the pieces was adjusted according to the experimental needs. The chip was immersed in 50 mM tris buffer of pH8.0 at 4° C. for later use.

### Example 2: In Situ Synthesis and Amplification of cDNA

1. Synthesis of cDNA

Mouse tissue sections were prepared according to the standard method of frozen sections, and the frozen sections were attached to the chip prepared in Example 1. After being fixed with frozen methanol for 30 minutes, the tissues were permeabilized using 0.5% triton x-100. 5×SSC was used to wash the chip twice at room temperature, 200 μL of the reverse transcriptase reaction system as shown in Table 3 was prepared, the reaction solution was added to the chip to fully cover it, and the reaction was carried out at 42° C. for 90 min to 180 min. The reverse transcriptase would use mRNA as a template to synthesize cDNA using a polyT-containing primer (its sequence was shown in SEQ ID NO: 6, which comprised a consensus sequence A (CA) and polyT sequence), and add a CCC overhang to the 3-end of the cDNA strand. After the TSO sequence (SEQ ID NO:7, which comprised a consensus sequence B (CB), UMI sequence (NNNNNN), and GGG sequence at the end) hybridized with and annealed to the cDNA strand (through complementary pairing of the GGG at the end of the TSO sequence and the CCC overhang of the cDNA strand), the reverse transcriptase would use the consensus sequence B and the UMI sequence as templates to continuously extending the cDNA strand, making a complementary sequence of the consensus sequence B and a complementary sequence of the UMI sequence carried at the 3-end of the cDNA. The chip was added with formamide solution and reacted at 55° C. for 5 minutes.

TABLE 3
Synthesis system of cDNA

| Ingredient | Volume (μL) | Final concentration | |
|---|---|---|---|
| Superscript II First strand buffer (5X) | 40 | 1X | |
| (Purchased from Thermo Fisher, Cat. No.: 18064022) | | | |
| Betaine (5M) | 40 | 1M | |
| (Purchased from Aladdin, Cat. No.: B105554) | | | |
| dNTP (10 mM) | 20 | 1 | mM |
| MgCl₂(100 mM) | 15 | 7.5 | mM |
| TSO sequence (SEQ ID NO: 7) | 10 | 1 | μM |
| (synthesized by Liuhe BGI) | | | |
| polyT primer (SEQ ID NO: 6) (comprising 5′-end | 10 | 1 | μM |
| phosphorylation modification) | | | |
| (Synthesized by Liuhe BGI) | | | |
| Superscript II RT (200 U/μL) | 10 | 10 | U/μL |
| (purchased from Thermo Fisher, Cat. No.: 18064022) | | | |
| DTT (100 mM) | 10 | 5 | mM |
| RNase inhibitor (40 U/μL) | 5 | 1 | U/μL |

| | | |
|---|---|---|
| (purchased from Thermo Fisher, Cat. No.: N8080119) | | |
| NF H₂O | 40 | — |

The synthesized cDNA strand comprised the following sequence structure: reverse transcription primer sequence (SEQ ID NO: 6) —cDNA sequence—c(TSO) sequence (complementary sequence of SEQ ID NO: 7).

2. Ligation Ofchip Sequence to cDNA Strand

Two kinds of bridging oligonucleotides (first bridging oligonucleotide and second bridging oligonucleotide, SEQ ID NOs: 9-10) comprising 5′-end phosphorylation modification were diluted to 100 μM with 2×SSC, and annealed at 30′ C for later use; 1 ml of the reaction solution shown in Table 4 was prepared, pumped at an appropriate volume into the chip to ensure that the chip was filled with the ligation reaction solution, and the reaction was carried out at room temperature for 30 minutes.

After the reaction was completed, the chip was washed with 5×SSC. 200 μL of Bst polymerization reaction solution (purchased from NEB, M0275) was prepared according to the instructions, pumped into the chip, and reacted at 65° C. for 60 minutes to obtain a double-stranded nucleic acid molecule comprising position information (i.e., tag sequence Y (Y) or its complementary sequence c(Y)), and one strand of which comprised the following sequence structure: cDNA sequence—complementary sequence of TSO sequence—first bridging oligonucleotide sequence—complementary sequence of partial sequence of chip sequence.

TABLE 4
Ligation system

| Ingredient | Volume (μL) | Final concentration | |
|---|---|---|---|
| 10X T4 ligase buffer | 100 | 1X | |
| (purchased from NEB, Cat. No.: M0202S) | | | |
| T4 ligase (600 U/μL) | 100 | 60 | U/μL |
| (purchased from NEB, Cat. No.: M0202S) | | | |
| Annealing product of first bridging oligonucleotide and | 10 | 1 | μM |

| | | |
|---|---|---|
| second bridging oligonucleotide (SEQ ID NOs: 9-10) | | |
| Glycerin | 10 | 10% |
| H₂O | 780 | — |

3. Release of cDNA

75 μL of 80 mM KOH was used to incubate the chip at room temperature for 5 minutes. After the resulting liquid was collected, 10 μL of 1M, pH8.0 Tris-HCl was added to neutralize the cDNA recovery solution.

4. Amplification of cDNA

200 μL of the reaction system as shown in Table 5 was prepared, used for transcriptome sequencing and library construction, and divided into 2 tubes for PCR:

TABLE 5
Amplification system of cDNA

| Ingredient | Volume (μL) | Final concentration | |
|---|---|---|---|
| cDNA recovery product | 84 | — | |
| Primer 1 (SEQ ID NO: 11) (synthesized by Liuhe BGI) | 8 | 0.8 | μM |
| Primer 2 (SEQ ID NO: 12) (synthesized by Liuhe BGI) | 8 | 0.8 | μM |

| | | |
|---|---|---|
| 2x HiFi (purchased from NEB, Cat. No.: E2621S) | 100 | 1X |

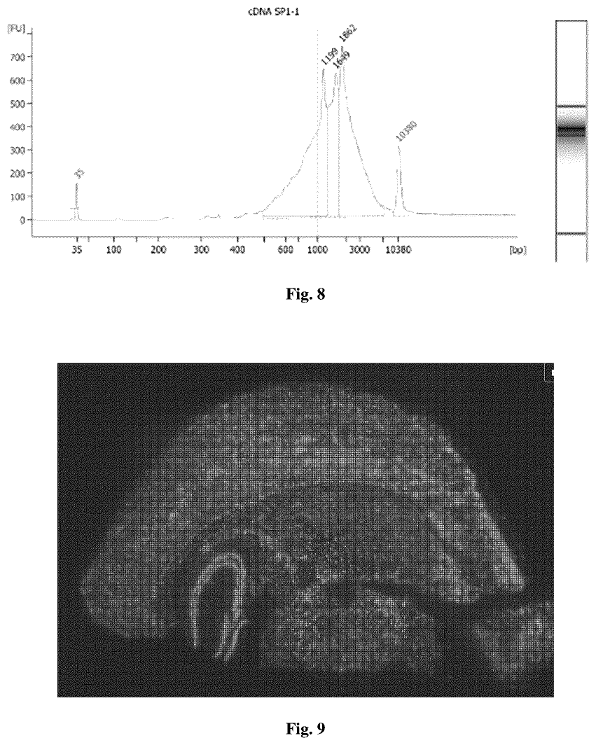

The above reaction system was placed into a PCR machine and the reaction program was set as follows: 95° C. 3 min, 11 cycles (98° C. 20 s, 58° C. 20 s, 72° C. 3 min), 72° C. 5 min, 4° C. ∞. After the reaction was completed, XP beads (purchased from AMPure) were used for magnetic bead-based purification and recovery. The dsDNA concentration was quantified using a Qubit kit, and the length distribution of the cDNA amplification product was detected using a 2100 Bioanalyzer (purchased from Agilent). The detection results were shown in FIG. 8.

### Example 3: Library Construction and Sequencing of cDNA

### 1. Tn5 Fragmentation

According to the cDNA concentration, 20 ng of cDNA (obtained in step 4 of Example 2) was taken, added with 0.5 μM of Tn5 transposase and corresponding buffer (purchased from BGI, Cat. No.: 10000028493; the coating of Tn5 transposase was operated according to the instruction of Stereomics library preparation kit-Si), mixed well to obtain a 20 μL reaction system, the reaction was carried out at 55° C. for 10 minutes, then 5 μL of 0.1% SDS was added and mixed well at room temperature for 5 minutes to terminate the Tn5 transposition.

### 2. PCR Amplification

100 μL of the following reaction system was prepared:

TABLE 6
Reaction system for library construction and amplification

| | | Final | |
|---|---|---|---|
| Ingredient | Volume (μL) | concentration | |
| Product after Tn5 transposition | 25 | - | |
| 2X Hifi ready mix | 50 | 0.8 | μM |
| Library construction primer 1 (10 μM, comprising | 4 | 0.4 | μM |
| phosphorylation modification at 5′-end, and | | | |
| phosphorothioate modification of the last two nucleotides | | | |
| at 3′-end) (SEQ ID NO: 13) (synthesized by Liuhe BGI) | | | |
| Library construction primer 2 (10 μM) (SEQ ID NO: 14) | 4 | 0.4 | μM |

| | | |
|---|---|---|
| (synthesized by Liuhe BGI) | | |
| NF H₂O | 17 | — |

After mixing, it was placed in a PCR machine and the program was set as follows: 95° C. 3 min, 11 cycles (98° C. 20 s, 58° C. 20 s, 72° C. 3 min), 72° C. 5 min, 4° C. ∞. After the reaction was completed, XP beads were used for magnetic bead-based purification and recovery. The dsDNA concentration was quantified using Qubit.

### 3. Sequencing

80 fmol of the above-fragmented amplification product was taken to prepare DNBs. 40 μL of reaction system was prepared as follows:

TABLE 7
DNB preparation system for sequencing

| Ingredient | Volume (μL) | Final concentration |
|---|---|---|
| Amplification product of step 2 above | X (80 fmol) | — |
| 10X phi29 buffer (obtained from Thermofisher, | 4 | 1X |
| Cat. No.: B62) | | |
| 10 μM primer for the preparation of DNBs for | 4 | 1 μM |
| sequencing (SEQ ID NO: 15) | | |
| H₂O | 32-x | — |

The above reaction volume was placed in a PCR machine for reaction, and the reaction conditions were as follows: 95° C. for 3 minutes, 40° C. for 3 minutes. After the reaction was completed, the resulting reaction solution was placed on ice and added with 40 μL of the mixed enzyme I required for DNB preparation in the DNBSEQ sequencing kit, 2 μL of the mix enzyme II, 1 μL of ATP, and 0.1 μL of T4 ligase. After mixing, the above reaction system was placed in a PCR machine and reacted at 30° C. for 20 minutes to form DNBs.

According to the method described in the PE50 kit for supporting MGISEQ 2000, the DNBs were loaded onto the sequencing chip of MGISEQ2000, and sequencing was performed according to the relevant instructions. PE self-defined sequencing model was selected, in which the first-strand sequencing was divided into two sections of sequencing, and 25 bp was sequenced first and then underwent 36 cycles of dark reaction, then 6 bp UMI sequence was sequenced, and the second-strand sequencing was set to sequence 50 bp.

### 4. Results

(1) After logging into the website http://stereomap.cngb.org/Stereo-Draftsman/report/index, data analysis was carried out according to the website operation guide. The first 25 bp of the read1 sequence (from first-strand sequencing) obtained by sequencing in step 3 was compared with the 25 bp position information in the chip preparation process in Example 1, and the reads aligned well with the position information on the chip were retained, and mapped to the corresponding chip positions. The reads2 (from second-strand sequencing) corresponding to the reads corresponding to the chip positions were found out, the reads2 were compared with the mouse brain genome, and duplicate reads were removed based on the UMI information, thereby obtaining the expression level of each gene in the mouse brain.

(2) The expression level of each gene was used for plotting, thereby obtaining the spatial gene expression map of mouse brain section as shown in FIG. 9.

The results in FIG. 9 showed that the method based on the present application could determine the spatial expression of genes in tissue samples with high throughput.

Although the specific embodiments of the present application have been described in detail, those skilled in the art will understand that various modifications and changes can be made to the details based on all teachings that have been disclosed, and these changes are all within the protection scope of the present application. The full scope of the present application is given by the appended claims and any equivalents thereof.

What is claimed is:

1. A method for generating a population of labeled nucleic acid molecules, which comprises the following steps:
(1) providing a biological sample and a nucleic acid array, wherein the nucleic acid array comprises a solid support, the solid support is coupled with multiple kinds of oligonucleotide probes, each kind of oligonucleotide probe comprises at least one copy; and the oligonucleotide probe comprises or consists of a consensus sequence X1, a tag sequence Y, and a consensus sequence X2 in the direction from 5′ to 3′, wherein,
each kind of oligonucleotide probe has a different tag sequence Y, and the tag sequence Y has a nucleotide sequence unique to the position of the kind of oligonucleotide probe on the solid support;
(2) contacting the biological sample with the nucleic acid array so that the position of an RNA (e.g., mRNA) in the biological sample is mapped to the position of the oligonucleotide probe on the nucleic acid array; preprocessing the RNA (e.g., mRNA) in the biological sample to generate a first nucleic acid molecule population, wherein the preprocessing comprises:
(i) (a) using a primer A to perform reverse transcription of the RNA (e.g., mRNA) of the biological sample to generate a cDNA strand, the cDNA strand comprises a cDNA sequence that is complementary to the RNA (e.g., mRNA) and formed by reverse transcription primed by the primer A, and a 3′-end overhang; wherein, the primer A comprises a capture sequence A, the capture sequence A is capable of annealing to an RNA (e.g., mRNA) to be captured and initiating an extension reaction; and, (b) annealing a primer B to the cDNA strand generated in (a), and performing an extension reaction to generate a first extension product as a first nucleic acid molecule to be labeled, thereby generating a first nucleic acid molecule population; wherein, the primer B comprises a consensus sequence B, a complementary sequence of the 3′-end overhang, and optionally a tag sequence B; the complementary sequence of the 3′-end overhang is located at the 3′-end of the primer B; the consensus sequence B is located upstream of the complementary sequence of the 3′-end overhang (e.g., located at the 5′-end of the primer B); or,
(ii) (a) using a primer A′ to perform reverse transcription of the RNA (e.g., mRNA) of the biological sample to generate a cDNA strand; the cDNA strand comprising a cDNA sequence that is complementary to the RNA (e.g., mRNA) and formed by reverse transcription primed by the primer A′, and a 3′-end overhang; wherein, the primer A′ comprises a consensus sequence A and a capture sequence A, the capture sequence A is capable of annealing to an RNA (e.g., mRNA) to be captured and initiating an extension reaction; the consensus sequence A is located upstream of the capture sequence A (e.g., located at the 5′-end of the primer A′); (b) annealing a primer B′ to the cDNA strand generated in (a) and performing an extension reaction to generate a first extension product; wherein, the primer B′ comprises a consensus sequence B, a complementary sequence of the 3′-end overhang, and optionally a tag sequence B; the complementary sequence of the 3′-end overhang is located at the 3′-end of the primer B′; the consensus sequence B is located upstream of the complementary sequence of the 3′-end overhang (e.g., located at the 5′-end of the primer B′); and, (c) providing an extension primer to perform an extension reaction using the first extension product as a template to generate a second extension product as a first nucleic acid molecule to be labeled, thereby generating a first nucleic acid molecule population;
(3) generating a second nucleic acid molecule population from the first nucleic acid molecule population obtained in the previous step by a step selected from the following:
(i) annealing (e.g., in-situ annealing) the oligonucleotide probe to the first nucleic acid molecule to be labeled which is at the corresponding position of the oligonucleotide probe, by applying a annealing condition to the product of step (2), and performing an extension reaction to generate an extension product as a second nucleic acid molecule with a positioning tag, thereby generating a second nucleic acid molecule population; wherein the consensus sequence X2 or partial sequence thereof of the oligonucleotide probe is (a) capable of annealing to a complementary sequence or partial sequence thereof of the consensus sequence B of the first extension product obtained in step (2)(i), or, (b) capable of annealing to a complementary sequence or partial sequence thereof of the consensus sequence A of the second extension product obtained in step (2)(ii); or,
(ii) contacting a bridging oligonucleotide pair with the oligonucleotide probe and the first nucleic acid molecule population obtained in the previous step under a condition that allows annealing, annealing (e.g., in-situ annealing) the bridging oligonucleotide pair to the oligonucleotide probe and the first nucleic acid molecule to be labeled which is at the corresponding position of the oligonucleotide probe,
wherein, the bridging oligonucleotide pair is composed of a first bridging oligonucleotide and a second bridging oligonucleotide, and the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; and the second region of the first bridging oligonucleotide is capable of annealing to the consensus sequence X2 or partial sequence thereof of the oligonucleotide probe;
the second region of the second bridging oligonucleotide is (a) capable of annealing to a complementary sequence or partial sequence thereof of the consensus sequence B of the first extension product obtained in step (2)(i), or, (b) capable of annealing to a complementary sequence or partial sequence thereof of the consensus sequence A of the second extension product obtained in step (2)(ii);
wherein, among the bridging oligonucleotide pair to be contacted with the first nucleic acid molecule population and the oligonucleotide probe, the first bridging oligonucleotide and the second bridging oligonucleotide of the bridging oligonucleotide pair each exist in a single-stranded form, or, the first bridging oligonucleotide and the second bridging oligonucleotide of the bridging oligonucleotide pair are annealed to each other and exist in a partial double-stranded form;
performing a ligation reaction to ligate the nucleic acid molecule hybridized with the first region and the nucleic acid molecule hybridized with the second region of the same first bridging oligonucleotide, and/or, to ligate the nucleic acid molecule hybridized with the first region and the nucleic acid molecule hybridized with the second region of the same second bridging oligonucleotide; and performing an extension reaction to obtain a reaction product as a second nucleic acid molecule with a positioning tag, thereby generating a second nucleic acid molecule population; wherein, the ligation reaction and the extension reaction are performed in any order.

2. The method according to claim 1, wherein in step (3)(ii):
(1) the first region and the second region of the first bridging oligonucleotide are directly adjacent, the ligation of the nucleic acid molecule hybridized with the first region and the nucleic acid molecule hybridized with the second region of the same first bridging oligonucleotide comprises: using a nucleic acid ligase to ligate the nucleic acid molecule hybridized with the first region and the nucleic acid molecule hybridized with the second region of the same first bridging oligonucleotide; or,
the first bridging oligonucleotide comprises the first region, the second region and the third region between them, the ligation of the nucleic acid molecule hybridized with the first region and the nucleic acid molecule hybridized with the second region of the same first bridging oligonucleotide comprises: using a nucleic acid polymerase (e.g., a nucleic acid polymerase without 5′ to 3′ exonucleolytic activity or strand displacement activity) to perform a polymerization reaction with the third region as a template, and using a nucleic acid ligase to ligate the nucleic acid molecule hybridized with the first region and the nucleic acid molecule hybridized with the third region and the second region of the same first bridging oligonucleotide;
and/or
(2) the first region and the second region of the second bridging oligonucleotide are directly adjacent, the ligation of the nucleic acid molecule hybridized with the first region and the nucleic acid molecule hybridized with the second region of the same second bridging oligonucleotide comprises: using a nucleic acid ligase to ligate the nucleic acid molecule hybridized with the first region and the nucleic acid molecule hybridized with the second region of the same second bridging oligonucleotide; or,
The second bridging oligonucleotide comprises the first region, the second region and the third region between them, the ligation of the nucleic acid molecule hybridized with the first region and the nucleic acid molecule hybridized with the second region of the same second bridging oligonucleotide comprises: using a nucleic acid polymerase (e.g., a nucleic acid polymerase without 5′ to 3′ exonucleolytic activity or strand displacement activity) to perform a polymerization reaction with the third region as a template, and using a nucleic acid ligase to ligate the nucleic acid molecule hybridized with the first region and the nucleic acid molecule hybridized with the third region and the second region of the same second bridging oligonucleotide.

3. The method according to claim 1 or 2, which comprises step (1), step (2)(i) and step (3); wherein, in step (2)(i)(b), the primer B comprises the consensus sequence B, a complementary sequence of the 3′-end overhang, and the tag sequence B;
preferably, in step (3), the second nucleic acid molecule derived from each copy of the same kind of oligonucleotide probe has a different tag sequence B as a UMI.

4. The method according to claim 3, which comprises step (1), step (2)(i) and step (3)(i); wherein the consensus sequence X2 or partial sequence thereof is capable of annealing to a complementary sequence or partial sequence thereof of the consensus sequence B; the extension product obtained in step (3)(i) is a labeled nucleic acid molecule, which comprises: a first strand comprising the sequence of the first nucleic acid molecule to be labeled, and/or, a second strand comprising the sequence of the oligonucleotide probe.

5. The method according to claim 4, wherein the consensus sequence X2 or partial sequence thereof is capable of annealing to a complementary sequence or partial sequence thereof of the consensus sequence B, and the complementary sequence of the consensus sequence B of the first extension product in step (2)(i) has a free 3′ end; wherein, the extension product obtained in step (3)(i) is the labeled nucleic acid molecule, which comprises the first strand;
preferably, in step (3)(i), the oligonucleotide probe is incapable of initiating an extension reaction (e.g., the 3′-end of the oligonucleotide probe is blocked).

6. The method according to claim 5, wherein in step (2)(i)(a), the capture sequence A of the primer A is a random oligonucleotide sequence.

7. The method according to claim 5, wherein in step (2)(i)(a), the capture sequence A of the primer A is a poly(T) sequence or a specific sequence targeting a target nucleic acid;
preferably, the primer A also comprises the consensus sequence A, and optionally a tag sequence A, such as a random oligonucleotide sequence.

8. The method according to claim 4, wherein the consensus sequence X2 or partial sequence thereof is capable of annealing to the complementary sequence or partial sequence thereof of the consensus sequence B, and the consensus sequence X2 of the oligonucleotide probe has a free 3′ end; wherein the extension product obtained in step (3)(i) is the labeled nucleic acid molecule, which comprises the second strand;
preferably, the first extension product obtained in step (2)(i) is incapable of initiating an extension reaction (e.g., the 3′-end of the first extension product obtained in step (2)(i) is blocked).

9. The method according to claim 8, wherein in step (2)(i)(a), the capture sequence A of the primer A is a random oligonucleotide sequence.

10. The method according to claim 8, wherein in step (2)(i)(a), the capture sequence A of the primer A is a poly(T) sequence or a specific sequence targeting a target nucleic acid;
preferably, the primer A also comprises the consensus sequence A, and optionally a tag sequence A, such as a random oligonucleotide sequence.

11. The method according to claim 3, which comprises step (1), step (2)(i) and step (3)(ii); wherein the second region of the second bridging oligonucleotide is capable of annealing to a complementary sequence or partial sequence thereof of the consensus sequence B of the first extension product obtained in step (2)(i); wherein, the reaction product obtained in step (3)(ii) is the labeled nucleic acid molecule, which comprises: a first strand comprising the sequence of the first nucleic acid molecule to be labeled, and/or a second strand comprising the sequence of the oligonucleotide probe.

12. The method according to claim 11, wherein the second region of the second bridging oligonucleotide is capable of annealing to the complementary sequence or partial sequence thereof of the consensus sequence B of the first extension product obtained in step (2)(i), and the second region of the first bridging oligonucleotide has a free 3′ end; wherein, the reaction product obtained in step (3)(ii) is the labeled nucleic acid molecule, which comprises the first strand;
preferably, the first bridging oligonucleotide has one or more of the following characteristics: i) the second region of the first bridging oligonucleotide is located at the 3′-end of the first bridging oligonucleotide; ii) the first region of the first bridging oligonucleotide is located at the 5′-end of the first bridging oligonucleotide; iii) the first bridging oligonucleotide comprises a 5′ phosphate at the 5′-end; iv) the first bridging oligonucleotide comprises a free —OH at the 3′-end;
preferably, the second bridging oligonucleotide is incapable of initiating an extension reaction (e.g., the 3′-end of the second bridging oligonucleotide is blocked), and/or the oligonucleotide probe is incapable of initiating an extension reaction (e.g., the 3′-end of the oligonucleotide probe is blocked).

13. The method according to claim 12, wherein in step (2)(i)(a), the capture sequence A of the primer A is a random oligonucleotide sequence.

14. The method according to claim 12, wherein in step (2)(i)(a), the capture sequence A of the primer A is a poly(T) sequence or a specific sequence targeting a target nucleic acid;
preferably, the primer A also comprises the consensus sequence A, and optionally a tag sequence A, such as a random oligonucleotide sequence.

15. The method according to claim 11, wherein the second region of the second bridging oligonucleotide is capable of annealing to the complementary sequence or partial sequence thereof of the consensus sequence B of the first extension product obtained in step (2)(i), and the second region of the second bridging oligonucleotide has a free 3′ end; wherein the reaction product obtained in step (3)(ii) is the labeled nucleic acid molecule, which comprises the second strand;
preferably, the second bridging oligonucleotide has one or more of the following characteristics: i) the second region of the second bridging oligonucleotide is located at the 3′-end of the second bridging oligonucleotide; ii) the first region of the second bridging oligonucleotide is located at the 5′-end of the second bridging oligonucleotide; iii) the second bridging oligonucleotide comprises a 5′ phosphate at the 5′-end; iv) the second bridging oligonucleotide comprises a free —OH at the 3′-end;
preferably, the first bridging oligonucleotide is incapable of initiating an extension reaction (e.g., the 3′-end of the first bridging oligonucleotide is blocked), and/or the first extension product obtained in step (2)(i) is incapable of initiating an extension reaction (e.g., the 3′-end of the first extension product obtained in step (2)(i) is blocked).

16. The method according to claim 15, wherein in step (2)(i)(a), the capture sequence A of the primer A is a random oligonucleotide sequence.

17. The method according to claim 15, wherein in step (2)(i)(a), the capture sequence A of the primer A is a poly(T) sequence or a specific sequence targeting a target nucleic acid;
preferably, the primer A also comprises the consensus sequence A, and optionally a tag sequence A, such as a random oligonucleotide sequence.

18. The method according to claim 1 or 2, which comprises step (1), step (2)(ii) and step (3);
wherein, in step (2)(ii)(b), the first extension product comprises from 5′ to 3′: the consensus sequence A, a cDNA sequence that is complementary to the RNA and formed by reverse transcription primed by the primer A′, the 3′-end overhang sequence, optionally a complementary sequence of the tag sequence B, and a complementary sequence of the consensus sequence B;
preferably, in step (2)(ii)(c), the extension primer is the primer B′ or a primer B″ or a random primer, wherein the primer B″ is capable of annealing to the complementary sequence or partial sequence thereof of the consensus sequence B, and capable of initiating the extension reaction.

19. The method according to claim 18, which comprises step (1), step (2)(ii) and step (3)(i); wherein the consensus sequence X2 or partial sequence thereof is capable of annealing to a complementary sequence or partial sequence thereof of the consensus sequence A; wherein, the extension product obtained in step (3)(i) is the labeled nucleic acid molecule, which comprises: a first strand comprising the sequence of the first nucleic acid molecule to be labeled, and/or, a second strand comprising the sequence of the oligonucleotide probe.

20. The method according to claim 19, wherein the consensus sequence X2 or partial sequence thereof is capable of annealing to the complementary sequence or partial sequence thereof of the consensus sequence A; wherein, the extension product obtained in step (3)(i) is the labeled nucleic acid molecule, which comprises the first strand comprising the sequence of the first nucleic acid molecule to be labeled;
preferably, in step (3)(i), the oligonucleotide probe is incapable of initiating an extension reaction (e.g., the 3′-end of the oligonucleotide probe is blocked).

21. The method according to claim 20, wherein in step (2)(ii)(a), the capture sequence A of the primer A′ is a random oligonucleotide sequence;
preferably, in step (3), the first strand derived from each copy of the same kind of oligonucleotide probe has a different complementary sequence of the capture sequence A as a UMI.

22. The method according to claim 20, wherein in step (2)(ii)(a), the capture sequence A of the primer A′ is a poly(T) sequence or a specific sequence targeting a target nucleic acid;
wherein the primer A′ also comprises a tag sequence A, such as a random oligonucleotide sequence;
preferably, in step (3), the first strand derived from each copy of the same kind of oligonucleotide probe has a different complementary sequence of the tag sequence A as a UMI.

23. The method according to claim 19, wherein the consensus sequence X2 or partial sequence thereof is capable of annealing to the complementary sequence or partial sequence thereof of the consensus sequence A; wherein, the extension product obtained in step (3)(i) is the labeled nucleic acid molecule, which comprises a second strand comprising the sequence of the oligonucleotide probe;
preferably, the second extension product obtained in step (2)(ii) is incapable of initiating an extension reaction (e.g., the 3′-end of the second extension product obtained in step (2)(ii) is blocked).

24. The method according to claim 23, wherein in step (2)(ii)(a), the capture sequence A of the primer A′ is a random oligonucleotide sequence;
preferably, in step (3), the second strand derived from each copy of the same kind of oligonucleotide probe has a different capture sequence A as a UMI.

25. The method according to claim 23, wherein in step (2)(ii)(a), the capture sequence A of the primer A′ is a poly(T) sequence or a specific sequence targeting a target nucleic acid; wherein, the primer A′ also comprises a tag sequence A, such as a random oligonucleotide sequence;
preferably, in step (3), the second strand derived from each copy of the same kind of oligonucleotide probe has a different tag sequence A as a UMI.

26. The method according to claim 18, which comprises step (1), step (2)(ii) and step (3)(ii); wherein the second region of the second bridging oligonucleotide is capable of annealing to the complementary sequence or partial sequence thereof of the consensus sequence A of the second extension product obtained in step (2)(ii); wherein, the reaction product obtained in step (3)(ii) is the labeled nucleic acid molecule, which comprises: the first strand comprising the sequence of the first nucleic acid molecule to be labeled, and/or, the second strand comprising the sequence of the oligonucleotide probe.

27. The method according to claim 20, wherein the second region of the second bridging oligonucleotide is capable of annealing to the complementary sequence or partial sequence thereof of the consensus sequence A of the second extension product obtained in step (2)(ii), and the second region of the first bridging oligonucleotide has a free 3′ end; wherein, the reaction product obtained in step (3)(ii) is the labeled nucleic acid molecule, which comprises the first strand;
preferably, the first bridging oligonucleotide has one or more of the following characteristics: i) the second region of the first bridging oligonucleotide is located at the 3′-end of the first bridging oligonucleotide; ii) the first region of the first bridging oligonucleotide is located at the 5′-end of the first bridging oligonucleotide; iii) the first bridging oligonucleotide comprises a 5′ phosphate at the 5′-end; iv) the first bridging oligonucleotide comprises a free —OH at the 3′-end;
preferably, the second bridging oligonucleotide is incapable of initiating an extension reaction (e.g., the 3′-end of the second bridging oligonucleotide is blocked), and/or, the oligonucleotide probe is incapable of initiating an extension reaction (e.g., the 3′-end of the oligonucleotide probe is blocked).

28. The method according to claim 27, wherein in step (2)(ii)(a), the capture sequence A of the primer A′ is a random oligonucleotide sequence;
preferably, in step (3), the first strand derived from each copy of the same kind of oligonucleotide probe has a different complementary sequence of the capture sequence A as a UMI.

29. The method according to claim 27, wherein in step (2)(ii)(a), the capture sequence A of the primer A′ is a poly(T) sequence or a specific sequence targeting a target nucleic acid; wherein, the primer A′ also comprises a tag sequence A, such as a random oligonucleotide sequence;
preferably, in step (3), the first strand derived from each copy of the same kind of oligonucleotide probe has a different complementary sequence of the tag sequence A as a UMI.

30. The method according to claim 26, wherein the second region of the second bridging oligonucleotide is capable of annealing to the complementary sequence or partial sequence thereof of the consensus sequence A of the second extension product obtained in step (2)(ii), and the second region of the second bridging oligonucleotide has a free 3′ end; wherein, the reaction product obtained in step (3)(ii) is the labeled nucleic acid molecule, which comprises the second strand;
preferably, the second bridging oligonucleotide has one or more of the following characteristics: i) the second region of the second bridging oligonucleotide is located at the 3′-end of the second bridging oligonucleotide; ii) the first region of the second bridging oligonucleotide is located at the 5′-end of the second bridging oligonucleotide; iii) the second bridging oligonucleotide comprises a 5′ phosphate at the 5′-end; iv) the second bridging oligonucleotide comprises a free —OH at the 3′-end;
preferably, the first bridging oligonucleotide is incapable of initiating an extension reaction (e.g., the 3′-end of the first bridging oligonucleotide is blocked), and/or the second extension product obtained in step (2)(ii) is incapable of initiating an extension reaction (e.g., the 3′-end of the second extension product obtained in step (2)(ii) is blocked).

31. The method according to claim 30, wherein in step (2)(ii)(a), the capture sequence A of the primer A′ is a random oligonucleotide sequence;
preferably, in step (3), the second strand derived from each copy of the same kind of oligonucleotide probe has a different capture sequence A as a UMI.

32. The method according to claim 30, wherein in step (2)(ii)(a), the capture sequence A of the primer A′ is a poly(T) sequence or a specific sequence targeting a target nucleic acid; wherein, the primer A′ also comprises a tag sequence A, such as a random oligonucleotide sequence;
preferably, in step (3), the second strand derived from each copy of the same kind of oligonucleotide probe has a different tag sequence A as a UMI.

33. The method according to any one of claims 1 to 17, wherein, in step (2)(i)(b), the cDNA strand anneals through its 3′-end overhang to the primer B, and, under the presence of a polymerase (e.g., a DNA polymerase or reverse transcriptase), the cDNA strand is extended using the primer B as a template to generate the first extension product.

34. The method according to any one of claims 1 to 2, 18 to 32, wherein in step (2)(ii)(b), the cDNA strand anneals through its 3′-end overhang to the primer B′, and, under the presence of a nucleic acid polymerase (e.g., a DNA polymerase or reverse transcriptase), the cDNA strand is extended using the primer B′ as a template to generate the first extension product.

35. The method according to any one of claims 1 to 34, wherein the 3′-end overhang has a length of at least 1, at least 2, at least 3, at least 4, at least 5, at least 6, at least 7, at least 8, at least 9, at least 10 or more nucleotides.

36. The method according to any one of claims 1 to 35, wherein in step (2), the biological sample is permeabilized before the preprocessing.

37. The method according to any one of claims 1 to 36, wherein the biological sample is a tissue sample;
preferably, the tissue sample is a tissue section.

38. The method according to any one of claims 1 to 37, wherein the reverse transcription in step (2) is performed by using a reverse transcriptase;
preferably, the reverse transcriptase has terminal deoxynucleotidyl transferase activity;
preferably, the reverse transcriptase is capable of synthesizing a cDNA strand using an RNA (e.g., mRNA) as a template, and adding an overhang at the 3′-end of the cDNA strand.

39. The method according to any one of claims 1 to 38, wherein steps (2) and (3) have one or more characteristics selected from the following:
(1) the primer A, primer A′, primer B, primer B′, the first bridging oligonucleotide, and the second bridging oligonucleotide each independently comprise or consist of natural nucleotides (e.g., deoxyribonucleotides or ribonucleotides), modified nucleotides, non-natural nucleotides, or any combination thereof; preferably, the primer A and primer A′ are capable of initiating an extension reaction;
(2) the primer B comprises a modified nucleotide (e.g., locked nucleic acid); preferably, the primer B comprises one or more modified nucleotides (e.g., one or more locked nucleic acids) at the 3′-end;
(3) the primer B′ comprises a modified nucleotide (e.g., locked nucleic acid); preferably, the primer B′ comprises one or more modified nucleotides (e.g., one or more locked nucleic acids) at the 3′-end;
(4) the tag sequence A and the tag sequence B each independently have a length of 5 to 200 nt (e.g., 5 to 30 nt, 6 to 15 nt);
(5) the consensus sequence A and the consensus sequence B each independently have a length of 10 to 200 nt (e.g., 10 to 100 nt, 20 to 100 nt, 25 to 100 nt, 5 to 10 nt, 10 to 15 nt, 15 to 20 nt, 20 to 50 nt, 20 to 30 nt, 30 to 40 nt, 40 to 50 nt, 50 to 100 nt);
(6) the primer A, primer A′, primer B, and primer B′ each independently have a length of 4 to 200 nt (e.g., 5 to 200 nt, 15 to 230 nt, 26 to 115 nt, 10 to 130 nt, 10 to 20 nt, 20 to 50 nt, 20 to 30 nt, 30 to 40 nt, 40 to 50 nt, 50 to 100 nt, 100 to 150 nt, 150 to 200 nt);
(7) the first region and the second region of the first bridging oligonucleotide each independently have a length of 3 to 100 nt (e.g., 20 to 100 nt, 3 to 10 nt, 10 to 15 nt, 15 to 20 nt, 20 to 70 nt, 20 to 30 nt, 30 to 40 nt, 40 to 50 nt, 50 to 100 nt);
(8) the first region and the second region of the second bridging oligonucleotide each independently have a length of 3 to 100 nt (e.g., 20 to 100 nt, 3 to 10 nt, 10 to 15 nt, 15 to 20 nt, 20 to 70 nt, 20 to 30 nt, 30 to 40 nt, 40 to 50 nt, 50 to 100 nt);
(9) the third region of the first bridging oligonucleotide and the third region of the second bridging oligonucleotide each independently have a length of 0 to 50 nt (e.g., 0 nt, 0 to 10 nt, 10 to 15 nt, 15 to 20 nt, 20 to 30 nt, 30 to 40 nt, 40 to 50 nt);
(10) the first bridging oligonucleotide and the second bridging oligonucleotide each independently have a length of 6 to 200 nt (e.g., 20 to 100 nt, 20 to 70 nt, 6 to 15 nt, 15 to 20 nt, 20 to 30 nt, 30 to 40 nt, 40 to 50 nt, 50 to 100 nt, 100 to 150 nt, 150 to 200 nt);
(11) the poly(T) sequence comprises at least 5, or at least 20 (e.g., 6 to 100, 10 to 50) deoxythymidine nucleoside residues;
(12) the random oligonucleotide sequence has a length of 5 to 200 (e.g., 5 nt, 5 to 30 nt, 6 to 15 nt).

40. The method according to any one of claims 1 to 39, wherein the method further comprises: (4) recovering and purifying the second nucleic acid molecule population.

41. The method according to any one of claims 1 to 40, wherein the obtained second nucleic acid molecule population and/or complement thereof are used for constructing a transcriptome library or for transcriptome sequencing.

42. The method according to any one of claims 1 to 41, wherein the oligonucleotide probe in step (1) has one or more characteristics selected from the following:
(1) the consensus sequence X1, tag sequence Y, and consensus sequence X2 each independently comprise or consist of natural nucleotides (e.g., deoxyribonucleotides or ribonucleotides), modified nucleotides, non-natural nucleotides (e.g., peptide nucleic acid (PNA) or locked nucleic acid), or any combination thereof;
(2) the consensus sequence X1, tag sequence Y, and consensus sequence X2 each independently have a length of 2 to 200 nt (e.g., 10 to 200 nt, 25 to 100 nt, 10 to 30 nt, 10 to 100 nt, 5 to 10 nt, 10 to 15 nt, 15 to 20 nt, 20-30 nt, 30-40 nt, 40-50 nt, 50-100 nt).

43. The method according to any one of claims 1 to 43, wherein the oligonucleotide probe is coupled to the solid support through a linker;
preferably, the linker is a linking group capable of coupling with an activating group, and the surface of the solid support is modified with the activating group;
preferably, the linker comprises —SH, -DBCO or —NHS;
preferably, the linker is -DBCO, and the surface of the solid support is modified with

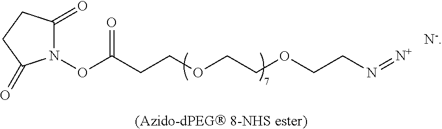

44. The method according to any one of claims 1 to 43, wherein the nucleic acid array in step (1) has one or more characteristics selected from the following:
(1) the oligonucleotide probes coupled to the same solid support have the same consensus sequence X1 and/or the same consensus sequence X2;
(2) the consensus sequence X1 of the oligonucleotide probe comprises a cleavage site; preferably, the cleavage site is capable of being cleaved or broken by means selected from nicking enzyme digestion, USER enzyme digestion, light-responsive excision, chemical excision, or CRISPR-mediated excision.

45. The method according to any one of claims 1 to 44, wherein the nucleic acid array in step (1) is provided by the following steps:
(1) providing a multiple kinds of carrier sequences, each kind of carrier sequence comprises at least one copy of the carrier sequence, and the carrier sequence in the direction from 5′ to 3′ comprises: a complementary sequence of the consensus sequence X2, a complementary sequence of the tag sequence Y, and an immobilization sequence; wherein, the complementary sequence of the tag sequence Y of each kind of carrier sequence is different from one another;
(2) attaching the multiple kind of carrier sequences to the surface of a solid support (e.g., a chip);
(3) providing an immobilization primer, and using the carrier sequence as a template to perform a primer extension reaction to generate an extension product, so as to obtain the oligonucleotide probe; wherein the immobilization primer comprises the sequence of the consensus sequence X1, and is capable of annealing to the immobilization sequence of the carrier sequence and initiating an extension reaction; preferably, the extension product in the direction from 5′ to 3′ comprises or consists of: the consensus sequence X1, the tag sequence Y and the consensus sequence X2;
(4) linking the immobilization primer to the surface of the solid support; wherein steps (3) and (4) are performed in any order;
(5) optionally, the immobilization sequence of the carrier sequence further comprises a cleavage site, and the cleavage can be selected from nicking enzyme digestion, USER enzyme digestion, light-responsive excision, chemical excision or CRISPR-mediated excision; performing cleavage at the cleavage site comprised in the immobilization sequence of the carrier sequence to digest the carrier sequence, so as to separate the extension product in step (3) from the template (i.e., the carrier sequence) from which the extension product is generated, thereby linking the oligonucleotide probe to the surface of the solid support (e.g., chip); preferably, the method further comprises separating the extension product in step (3) from the template (i.e., the carrier sequence) from which the extension product is generated through high-temperature denaturation;
preferably, each kind of carrier sequence is a DNB formed from a concatemer of multiple copies of the carrier sequence;
preferably, the multiple kinds of carrier sequences are provided in step (1) by the following steps:
(i) providing multiple kinds of carrier-template sequences, each carrier-template sequence comprises a complementary sequence of a carrier sequence;
(ii) using each kind of carrier-template sequence as a template to perform a nucleic acid amplification reaction so as to obtain an amplification product of each kind of carrier-template sequence, wherein the amplification product comprises at least one copy of the carrier sequence; preferably, rolling circle replication is performed to obtain a DNB formed from a concatemer of the carrier sequence.

46. The method according to any one of claims 1 to 45, wherein the solid support in step (1) has one or more characteristics selected from the following:
(1) the solid support is selected from the group consisting of latex bead, dextran bead, polystyrene surface, polypropylene surface, polyacrylamide gel, gold surface, glass surface, chip, sensor, electrode and silicon wafer; preferably, the solid support is a chip;
(2) the solid support is planar, spherical or porous;
(3) the solid support is capable of being used as a sequencing platform, such as a sequencing chip; preferably, the solid support is a sequencing chip for Illumina, MGI or Thermo Fisher sequencing platform; and
(4) the solid support is capable of releasing the oligonucleotide probe spontaneously or upon exposure to one or more stimuli (e.g., temperature change, pH change, exposure to specific chemicals or phases, exposure to light, exposure to reducing agents, etc.).

47. A method for constructing a library of nucleic acid molecules, which comprises:
(a) generating a population of labeled nucleic acid molecules according to the method according to any one of claims 1 to 46;
(b) randomly fragmenting the nucleic acid molecules in the population of labeled nucleic acid molecules and linking an adapter thereto; and
(c) optionally, amplifying and/or enriching the product of step (b);
thereby obtaining the library of nucleic acid molecules;
preferably, the library of nucleic acid molecules is used for sequencing, such as transcriptome sequencing, such as single cell transcriptome sequencing (e.g., 5′ or 3′ transcriptome sequencing).

48. The method according to claim 47, wherein, before performing step (b), the method further comprises step (pre-b): amplifying and/or enriching the population of labeled nucleic acid molecules;
preferably, the amplification reaction is performed using at least a primer C and/or a primer D, wherein the primer C is capable of hybridizing with or annealing to a complementary sequence or partial sequence thereof of the consensus sequence X1, and initiating an extension reaction; the primer D is capable of hybridizing with or annealing to the nucleic acid molecule comprising the tag sequence Y in the population of labeled nucleic acid molecules, and initiating an extension reaction.

49. The method according to claim 47 or 48, wherein in step (b), by using a transposase, the nucleic acid molecules obtained in the previous step are randomly fragmented and the resulting fragments are linked with an adapter at both ends;
preferably, in step (c), the product of step (b) is amplified using at least a primer C′ and/or a primer D′, wherein, the adapters at both ends of the fragment are a first adapter and a second adapter, the primer C′ is capable of hybridizing with or annealing to the first adapter, and initiating an extension reaction, and the primer D′ is capable of hybridizing with or annealing to the second adapter, and initiating an extension reaction.

50. A method for transcriptome sequencing of a cell in a sample, which comprises:
(1) constructing a library of nucleic acid molecules according to the method according to any one of claims 47 to 49; and
(2) sequencing the library of nucleic acid molecules.

51. A kit, which comprises:
(i) a nucleic acid array for labeling nucleic acids, which comprises a solid support, in which the solid support is coupled with multiple kinds of oligonucleotide probes; each kind of oligonucleotide probe comprises at least one copy; and, the oligonucleotide probe in the direction from 5′ to 3′ comprises or consists of: a consensus sequence X1, a tag sequence Y, and a consensus sequence X2, wherein,
each kind of oligonucleotide probe has a different tag sequence Y, and the tag sequence Y has a nucleotide sequence unique to the position of the oligonucleotide probe on the solid support;
(ii) a primer set comprising a primer A and a primer B or comprising a primer A′ and a primer B′, wherein:
the primer A comprises a capture sequence A, in which the capture sequence A is capable of annealing to an RNA (e.g., mRNA) to be captured and initiating an extension reaction;
the primer B comprises a consensus sequence B, a complementary sequence of a 3′-end overhang, and optionally a tag sequence B; wherein, the complementary sequence of a 3′-end overhang is located at the 3′-end of the primer B, the consensus sequence B is located upstream of the complementary sequence of a 3′-end overhang (e.g., located at the 5′-end of the primer B); wherein, the 3′-end overhang refers to one or more non-templated nucleotides comprised in the 3′-end of a cDNA strand generated by reverse transcription using an RNA captured by the capture sequence A of the primer A as a template;
the primer A′ comprises a consensus sequence A and a capture sequence A; wherein, the capture sequence A is located at the 3′-end of the primer A′, the consensus sequence A is located upstream of the capture sequence A (e.g., located at the 5′-end of the primer A′);
the primer B′ comprises a consensus sequence B, a complementary sequence of a 3′-end overhang, and optionally a tag sequence B; wherein, the complementary sequence of a 3′-end overhang is located at the 3′-end of the primer B′, the consensus sequence B is located upstream of the complementary sequence of a 3′-end overhang (e.g., located at the 5′-end of the primer B′); wherein, the 3′-end overhang refers to one or more non-templated nucleotides comprised in the 3′-end of a cDNA strand generated by reverse transcription using an RNA captured by the capture sequence A of the primer A′ as a template.

52. The kit according to claim 51, which comprises: the nucleic acid array for labeling nucleic acids as described in (i), the primer set comprising the primer A and primer B as described in (ii), and, (iii) a first bridging oligonucleotide and a second bridging oligonucleotide; wherein the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence B of the primer B;
wherein, the capture sequence A of the primer A is a random oligonucleotide sequence; or the capture sequence A of the primer A is a poly(T) sequence or a sequence specific for a target nucleic acid, the primer A preferably further comprises a consensus sequence A and optionally a tag sequence A, such as a random oligonucleotide sequence;
wherein, the primer B comprises a consensus sequence B, a complementary sequence of the 3′-end overhang, and a tag sequence B;
preferably, the primer B comprises a modified nucleotide (e.g., locked nucleic acid); preferably, the primer B comprises one or more modified nucleotides (e.g., one or more locked nucleic acids) at the 3′-end.

53. The kit according to claim 52, wherein the second region of the second bridging oligonucleotide is capable of annealing to a complementary sequence or partial sequence thereof of the consensus sequence B of the primer B;
preferably, the first bridging oligonucleotide has one or more of the following characteristics: i) the second region of the first bridging oligonucleotide is located at the 3′-end of the first bridging oligonucleotide; ii) the first region of the first bridging oligonucleotide is located at the 5′-end of the first bridging oligonucleotide; iii) the first bridging oligonucleotide comprises a 5′ phosphate at the 5′-end; iv) the first bridging oligonucleotide comprises a free —OH at the 3′-end;
preferably, the second bridging oligonucleotide is incapable of initiating an extension reaction (e.g., the 3′-end of the second bridging oligonucleotide is blocked), and/or the oligonucleotide probe is incapable of initiating an extension reaction (e.g., the 3′-end of the oligonucleotide probe is blocked).

54. The kit according to claim 52, wherein the second region of the second bridging oligonucleotide is capable of annealing to a complementary sequence or partial sequence thereof of the consensus sequence B of the primer B;
preferably, the second bridging oligonucleotide has one or more of the following characteristics: i) the second region of the second bridging oligonucleotide is located at the 3′-end of the second bridging oligonucleotide; ii) the first region of the second bridging oligonucleotide is located at the 5′-end of the second bridging oligonucleotide; iii) the second bridging oligonucleotide comprises a 5′ phosphate at the 5′-end; iv) the second bridging oligonucleotide comprises a free —OH at the 3′-end;
preferably, the first bridging oligonucleotide is incapable of initiating an extension reaction (e.g., the 3′-end of the first bridging oligonucleotide is blocked).

55. The kit according to claim 51, comprising: the nucleic acid array for labeling nucleic acids as described in (i), and the primer set comprising the primer A and primer B as described in (ii);
wherein, the capture sequence A of the primer A is a random oligonucleotide sequence; or, the capture sequence A of the primer A is a poly(T) sequence or a specific sequence targeting a target nucleic acid, and the primer A preferably further comprises a consensus sequence A and optionally a tag sequence A, such as a random oligonucleotide sequence;
wherein, the primer B comprises a consensus sequence B, a complementary sequence of the 3′-end overhang, and a tag sequence B;
preferably, the primer B comprises a modified nucleotide (e.g., locked nucleic acid); preferably, the primer B comprises one or more modified nucleotides (e.g., one or more locked nucleic acids) at the 3′ end.

56. The kit according to claim 51, which comprises: the nucleic acid array for labeling nucleic acids as described in (i), the primer set comprising the primer A′ and primer B′ as described in (ii), and, (iii) a first bridging oligonucleotide and a second bridging oligonucleotide; wherein the first bridging oligonucleotide and the second bridging oligonucleotide each independently comprise: a first region and a second region, and optionally a third region located between the first region and the second region, and the first region is located upstream of the second region (e.g., located 5′ of the second region); wherein,
the first region of the first bridging oligonucleotide is capable of annealing to the first region of the second bridging oligonucleotide; the second region of the first bridging oligonucleotide is capable of annealing to the consensus sequence X2 or partial sequence thereof of the oligonucleotide probe;
the second region of the second bridging oligonucleotide is capable of annealing to a complementary sequence or partial sequence thereof of the consensus sequence A of the primer A′;
wherein, the capture sequence A of the primer A′ is a random oligonucleotide sequence; or, the capture sequence A of the primer A′ is a poly(T) sequence or a specific sequence targeting a target nucleic acid, and the primer A′ further comprises a tag sequence A, such as a random oligonucleotide sequence;
preferably, the primer B′ comprises a modified nucleotide (e.g., locked nucleic acid); preferably, the primer B′ comprises one or more modified nucleotides (e.g., one or more locked nucleic acids) at the 3′-end;
preferably, the kit further comprises a primer B″ or a random primer, the primer B″ is capable of annealing to a complementary sequence or partial sequence thereof of the consensus sequence B, and capable of initiating an extension reaction.

57. The kit according to claim 56, wherein the second region of the second bridging oligonucleotide is capable of annealing to the complementary sequence or partial sequence thereof of the consensus sequence A of the primer A′;
preferably, the first bridging oligonucleotide has one or more of the following characteristics: i) the second region of the first bridging oligonucleotide is located at the 3′-end of the first bridging oligonucleotide; ii) the first region of the first bridging oligonucleotide is located at the 5′-end of the first bridging oligonucleotide; iii) the first bridging oligonucleotide comprises a 5′ phosphate at the 5′-end; iv) the first bridging oligonucleotide comprises a free —OH at the 3′-end;
preferably, the second bridging oligonucleotide is incapable of initiating an extension reaction (e.g., the 3′-end of the second bridging oligonucleotide is blocked), and/or the oligonucleotide probe is incapable of initiating an extension reaction (e.g., the 3′-end of the oligonucleotide probe is blocked).

58. The kit according to claim 56, wherein the second region of the second bridging oligonucleotide is capable of annealing to a complementary sequence or partial sequence thereof of the consensus sequence A of the primer A′;
preferably, the second bridging oligonucleotide has one or more of the following characteristics: i) the second region of the second bridging oligonucleotide is located at the 3′-end of the second bridging oligonucleotide; ii) the first region of the second bridging oligonucleotide is located at the 5′-end of the second bridging oligonucleotide; iii) the second bridging oligonucleotide comprises a 5′ phosphate at the 5′-end; iii) the second bridging oligonucleotide comprises a free —OH at the 3′-end;
preferably, the first bridging oligonucleotide is incapable of initiating an extension reaction (e.g., the 3′-end of the first bridging oligonucleotide is blocked).

59. The kit according to claim 51, which comprises: the nucleic acid array for labeling nucleic acids as described in (i), and the primer set comprising the primer A′ and primer B′ as described in (ii);
wherein, the capture sequence A of the primer A′ is a random oligonucleotide sequence; or, the capture sequence A of the primer A′ is a poly(T) sequence or a specific sequence targeting a target nucleic acid, and the primer A′ further comprises a tag sequence A, such as a random oligonucleotide sequence;
wherein, the primer B′ comprises a consensus sequence B, a complementary sequence of the 3′-end overhang, and a tag sequence B;
preferably, the primer B′ comprises a modified nucleotide (e.g., locked nucleic acid); preferably, the primer B′ comprises one or more modified nucleotides (e.g., one or more locked nucleic acids) at the 3′-end;
preferably, the kit further comprises a primer B″ or a random primer, the primer B″ is capable of annealing to a complementary sequence or partial sequence thereof of the consensus sequence B, and capable of initiating an extension reaction.

60. The kit according to any one of claims 51 to 59, which has one or more characteristics selected from the following:
(1) the oligonucleotide probe, primer A, primer A′, primer B, primer B′, primer B″, random primer, first bridging oligonucleotide, and second bridging oligonucleotide each independently comprise or consist of natural nucleotides (e.g., deoxyribonucleotides or ribonucleotides), modified nucleotides, non-natural nucleotides, or any combination thereof;
(2) the oligonucleotide probes each independently have a length of 15 to 300 nt (e.g., 15 to 200 nt, 15 to 20 nt, 20 to 30 nt, 30 to 40 nt, 40 to 50 nt, 50 to 100 nt, 100 to 150 nt, 150 to 200 nt);
(3) the primer A, primer A′, primer B, primer B′, primer B″, and random primer each independently have a length of 4 to 200 nt (e.g., 5 to 200 nt, 15 to 230 nt, 26 to 115 nt, 10 to 130 nt, 10 to 20 nt, 20 to 50 nt, 20 to 30 nt, 30 to 40 nt, 40 to 50 nt, 50 to 100 nt, 100 to 150 nt, 150 to 200 nt);
(4) the first bridging oligonucleotide and the second bridging oligonucleotide each independently have a length of 6 to 200 nt (e.g., 20 to 100 nt, 20 to 70 nt, 6 to 15 nt, 15 to 20 nt, 20 to 30 nt, 30 to 40 nt, 40 to 50 nt, 50 to 100 nt, 100 to 150 nt, 150 to 200 nt);
(5) the oligonucleotide probes coupled to the same solid support have the same consensus sequence X1 and/or the same consensus sequence X2;
(6) the consensus sequence X1 of the oligonucleotide probes comprises a cleavage site; preferably, the cleavage site is capable of being cleaved or broken by a method selected from nicking enzyme digestion, USER enzyme digestion, light-responsive excision, chemical excision or CRISPR-mediated excision.

61. The kit according to any one of claims 51 to 60, which further comprises a reverse transcriptase, a nucleic acid ligase, a nucleic acid polymerase and/or a transposase;
preferably, the reverse transcriptase has terminal deoxynucleotidyl transferase activity; preferably, the reverse transcriptase is capable of synthesizing a cDNA strand using an RNA (e.g., mRNA) as a template, and adding an overhang to the 3′-end of the cDNA strand.

62. The kit according to any one of claims 51 to 61, which further comprises: a reagent for nucleic acid hybridization, a reagent for nucleic acid extension, a reagent for nucleic acid amplification, a reagent for recovering or purifying nucleic acid, a reagent for constructing a library for transcriptome sequencing, a reagent for sequencing (e.g., second- or third-generation sequencing), or any combination thereof.

63. Use of the method according to any one of claims 1 to 46 or the kit according to any one of claims 51 to 62 for constructing a library of nucleic acid molecules or for performing transcriptome sequencing.
